(19) United States
(12) Patent Application Publication
(10) Pub. No.: US 2023/0390315 A1
(43) Pub. Date: Dec. 7, 2023

(54) COMPOSITIONS AND METHODS FOR TREATING ISCHEMIC CONDITIONS

(71) Applicant: The Regents of the University of California, Oakland, CA (US)

(72) Inventors: Napoleone Ferrara, San Diego, CA (US); Cuiling Zhong, San Diego, CA (US)

(21) Appl. No.: 18/249,515

(22) PCT Filed: Oct. 20, 2021
(86) PCT No.: PCT/US2021/055809

§ 371 Date: Apr. 18, 2023

Related U.S. Application Data
(60) Provisional application No. 63/094,032, filed on Oct. 20, 2020.

(51) Int. Cl. A61K 31/7008; A61K 38/18; A61P 9/10

(52) U.S. Cl. CPC A61K 31/7008; A61P 9/10; A61K 38/1866

(57) ABSTRACT

Compositions for treating an ischemic condition in a subject may include hexosamine D-mannosamine (ManN). Methods may include administering to the subject in need thereof an effective amount of ManN. The administration may be effective to promote endothelial cell proliferation and angiogenesis in the subject. The subject may be in need of induced angiogenesis due to an ischemic condition caused by disease or trauma. Compositions for inhibiting protein glycosylation in a cell may include ManN. Methods for inhibiting protein glycosylation in a cell may include administering to the cell an effective amount of ManN.

### CROSS REFERENCE TO RELATED APPLICATIONS

This application claims the priority benefit of U.S. Provisional Application No. 63/094,032, filed Oct. 20, 2020, which application is incorporated herein by reference in its entirety.

### TECHNICAL FIELD

The present disclosure generally relates to compositions and methods for treating ischemic conditions.

### BACKGROUND

Angiogenesis is a complex process involving the growth of new blood vessels from the existing vasculature and occurs in both physiological and pathological circumstances. In tumors, angiogenesis facilitates rapid growth and metastasis through delivery of nutrients and oxygen and removal of metabolic wastes [1]. Development of the vasculature requires the coordinated activation of multiple signaling pathways, including VEGF/VEGFR, angiopoietin (Ang)/Tie2, Notch, Ephrin/Eph and PDGF/PDGFR [2, 3]. Stimulating angiogenesis has the potential of facilitating treatment of a number of conditions characterized by reduced perfusion, including diabetic ulcers, myocardial and limb ischemia [4, 5]. Conversely, blocking angiogenesis is a clinically validated strategy to treat malignant tumors and intraocular neovascular disorders [1, 6].

Endothelial cell (EC) metabolism is hypothesized to play a key role in the regulation of angiogenesis in normal and pathological circumstances. Metabolic switches in ECs, such as fatty acid, glucose, and glutamine metabolism, have been reported to trigger angiogenesis [7, 8]. ECs in the tumor vasculature are known to rely on glycolysis for ATP production, for instance through enhanced expression of glucose transporter GLUT1. Lowering glycolysis in tumor ECs arrests their proliferation [9]. In addition, aberrant glycosylation patterns have been documented during oncogenic transformation and progression of cancer and it has been proposed that inhibiting glycosylation may result in suppression of key angiogenesis pathways, including VEGF/VEGFR2 and Notch [10]. Evidence that has emerged in recent years points to glycans as novel angiogenesis regulators due to changes in protein glycosylation [11]. For example, the glycan-binding protein Galectin1 has been reported to interact with VEGFR2, leading to ligand-independent receptor activation, which may contribute to tumor resistance to anti-VEGF therapy [11]. Therefore, EC metabolism has been identified as a new target for anti-angiogenic therapy, particularly through inhibition of energy metabolism and glycosylation.

### SUMMARY

The present disclosure provides pharmaceutical compositions and methods for treating an ischemic condition in a subject. In embodiments, a composition for treating an ischemic condition in a subject includes hexosamine D-mannosamine (ManN). In embodiments, a method for treating an ischemic condition in a subject includes administering to the subject in need thereof an effective amount of hexosamine D-mannosamine (ManN).

In embodiments, the administration is effective to promote endothelial cell proliferation and angiogenesis in the subject. In embodiments, the method further includes administering to the subject in need thereof an effective amount of an N-glycosylation inhibitor. In embodiments, the method further includes administering to the subject in need thereof an effective amount of VEGF.

In embodiments, the ischemic condition is caused by a disease or a trauma. In embodiments, the administration is intravenous, intraperitoneal, or intravitreal.

In embodiments, the present disclosure provides pharmaceutical compositions and methods for inducing angiogenesis in a subject, including administering to the subject in need thereof an effective amount of hexosamine D-mannosamine (ManN).

In embodiments, the administration is effective to reduce ischemia in the subject. In embodiments, the method further includes administering to the subject in need thereof an effective amount of an N-glycosylation inhibitor. In embodiments, the method further includes administering to the subject in need thereof an effective amount of VEGF.

In embodiments, the subject is in need of inducing angiogenesis due to an ischemic condition is caused by a disease or a trauma.

In embodiments, the administration is intravenous, intraperitoneal, or intravitreal.

In embodiments, the present disclosure provides pharmaceutical compositions and methods for inhibiting protein glycosylation in a cell, including administering to the cell an effective amount of hexosamine D-mannosamine (ManN).

In embodiments, the administration is in vivo. In embodiments, the administration is ex vivo. In embodiments, the administration is effective to stimulate EC proliferation and angiogenesis. In embodiments, the administration is effective to activate JNK and an unfolded protein response caused by ER stress. In embodiments, the administration is effective to induce changes in N-glycan and O-glycan profiles.

### BRIEF DESCRIPTION OF THE DRAWINGS

FIGS. 1A to 1F represent examples showing the effects of Mannosamine (ManN) on bovine choroidal microvascular endothelial cells (BCEC) proliferation. FIG. 1A is an image showing crystal-violet stained BCEC samples treated with ManN, in the presence or absence of VEGF. BCECs were treated with various concentrations of ManN ranging from 0.5 μM to 1 mM for 5-6 days, with or without 5 ng/ml VEGF. At the end of the experiment, cells were fixed and stained with crystal violet. Cell-covered areas in various treatment groups were quantified by ImageJ software. FIG. 1B is a chart showing the effect of ManN in the presence or absence of VEGF on cell numbers. Cell numbers were quantified by adding AlamarBlue and fluorescence was measured at 530 nm/590 nm. n=3 independent samples were used. FIG. 1C is a chart showing the effect of ManN on bovine retinal microvascular endothelial cells (BREC) proliferation. n=3 biologically independent samples were used. FIG. 1D is an image showing effects of hexosamines other than ManN on BCEC proliferation in samples. Each treatment group was tested in duplicate. FIG. 1E is a chart and images showing effect of ManN on wounded BCEC samples. BCEC confluent monolayers were scratched with 1 ml pipet tip, washed and then incubated for 40 hours in low glucose DMEM containing 1% FBS. n=3 independent samples were used. Scale bar=400 μm. Images were taken and gaps between leading wound front were quantified using AxioVision LE Re1.4.4 software. Representative images from crystal violet staining are shown. FIG. 1F is a chart showing effects of ManN in BCEC transwell migration assay. n=4 independent samples were used. Asterisks indicate a significant difference compared with control. When statistical analysis was done using a different control, a line was used between specific groups. A representative experiment is shown from 2 independent studies. Data are means+/−SD, Statistical analysis was done by 2-tailed, two-sample unequal variance t test. *p<0.05, **p<0.01.

FIGS. 2A to 2C are western blot images showing activation of ERK, AKT, mTOR, AMPKα, CREB, ACC, and eNOS is not unique to ManN. Enhanced activation of ERK (Thr 202/Tyr 204), AKT (Ser 473) and CREB (Ser 133) in BCECs following treatment with ManN together with VEGF for various times (FIG. 2A) or following pre-treatment with ManN for 8 hours, followed by VEGF stimulation for 15 minutes (FIG. 2B). For the samples shown in FIG. 2C, BCECs were treated with 40 μM ManN, ManN Ac or mannose for various times. Total mTOR, ACC, eNOS, AMPKα, ERK, AKT, CREB, as well as phosphorylation of mTOR (Ser 2448), ACC (Ser 79), eNOS (Ser 1177), AMPKα (Thr 172), ERK (Thr 202/Tyr 204), AKT (Ser 473), and CREB (Ser 133) were examined by western blot analysis. β-actin served as the loading control. Molecular weight (KDa) was labeled at the right. A representative experiment is shown from 2 independent studies.

FIGS. 3A to 3E represent examples showing that ManN specifically activates the JNK pathway in BCECs. FIG. 3A is a western blot image. BCECs grown in Growth Media (GM: low glucose DMEM containing 10% bovine calf serum (BCS), 10 ng/ml VEGF and bFGF) were switched to growth factor-free media, followed by treatment with ManN or Mannose at 4 μM-4 mM. Four hours later, cell lysates were collected and subjected to western blot analysis for phosphorylated JNK (Thr 183/Tyr 185), p38 (Thr 180/Tyr 182) and ERK (Thr 202/Tyr 204), as well as total JNK, p38 and ERK. FIG. 3B is a western blot image showing that ManN, but not mannose, could activate JNK and its downstream c-Jun. β-actin served as the loading control. For each study, a representative experiment is shown from 2-3 independent studies. FIG. 3C is a chart showing the effect of ManN on pre-treated samples. BCECs plated in 96-well plates were attached, pre-treated with the specific JNK inhibitor SP600125 (5 μM) for 2 hours, followed by ManN at either 40 μM or 2 mM, with or without 5 ng/ml VEGF. Six days later, cell proliferation was quantified using AlamarBlue. n=3 independent samples were used. FIG. 3D is a western blot image showing screening of siRNAs against JNK1 and JNK2. 24 hours after siRNA transfection, BCECs were lysed and proteins were subjected to western blot analysis. β-actin served as the loading control. Quantification of target knockdown is shown. FIG. 3E is a chart showing example results showing that ˜80% knockdown of JNK1 and/or JNK2 by two independent siRNAs was associated with a significant reduction in the stimulatory effects of ManN on BCEC proliferation. n=3 independent samples were used. Data are means+/−SD, Asterisks indicated a significant difference compared with the control. When statistical analysis was done using a different control, a line was used between specific groups. Statistical analysis was done by 2-tailed, two-sample unequal variance t test. *p<0.05, **p<0.01.

FIGS. 4A to 4G represent examples showing that ManN affects protein glycosylation. FIG. 4A is a western blot image showing reduction of VEGFR2 molecular mass following ManN treatment. BCECs were treated with various hexosamines, their derivatives and monosaccharides at 40 μM or with VEGF at 5 ng/ml for 24 hours. VEGFR2 western blot analysis was performed. FIG. 4B is a western blot imaging showing dose-dependent effects of ManN on VEGFR2 molecular mass in BCECs. FIG. 4C is a western blot image showing Mannose could dose-dependently reverse the effect of 2 mM ManN on VEGFR2 molecular mass change, whereas mannose alone had no effect even at 10 mM. FIG. 4D is a chart showing that 5 mM mannose could completely reverse the bell-shaped effects of ManN on BCEC proliferation with or without 5 ng/ml VEGF. BCECs plated in 96 wells were allowed to attach, followed by ManN addition. Two hours later, cells were treated with different concentrations of Mannose, with or without VEGF. Six days later, cell proliferation was quantified using AlamarBlue. n=3 independent samples were used. FIG. 4E is a western blot image showing that effects of ManN are reversible. BCECs, after treatment with 40 μM ManN for 24 hours, were washed three times with low glucose DMEM. Cells were kept in low glucose DMEM for additional 8 or 24 hours. VEGFR2 western blot analysis was performed. FIG. 4F is a western blot image showing reduction of molecular mass of VEGFR2, Neuropilin-1, CD31 and c-met in HUVEC following ManN treatment at various concentrations. FIG. 4G is a western blot image showing reduction of molecular mass of VEGFR2, (31 integrin and bFGFR1 in hDMVECs by ManN at various concentrations. β-actin served as the loading control. Data are means+/−SD, Asterisks denote a significant difference compared with the control. For each study, a representative experiment is shown from 2-5 independent studies. Statistical analysis was done by 2-tailed, two-sample unequal variance t test. *p<0.05, **p<0.01.

FIGS. 5A to 5D represent examples showing that ManN specifically induces expression of unfolded protein response (UPR) responsive proteins. FIG. 5A is a western blot image. BCECs were grown in Growth Media (GM) until ˜80% confluency. Media were changed to growth factor-free media containing 10% BCS in the presence or absence of 40 or 400 μM of ManN or mannose for various times. At the end of each incubation, cell lysates were collected, proteins were separated on 4-12% Bis-Tris gel for western blot analysis. FIG. is a western blot image for cells treated with various concentrations of ManN, mannose, 5 ng/ml VEGF or a combination of ManN and VEGF for 24 hours. Cell lysates were separated on NuPAGE 3-8% Tris-Acetate gel for western blot analysis. FIG. 5C is a western blot image showing that 4-PBA, but not TUDCA, could effectively block the induction of CHOP in BCECs, accompanied by a restoration of expression of transcription factor ATF-6 upon 400 μM ManN treatment. BCECs were pre-treated with 2 mM 4-PBA or 500 μM TUDCA, two chemical chaperons. Sixteen hours later, cells were switched to growth factor-free media for 4 hours in the presence of ManN. GM: Growth Media. FIG. 5(d) 4-PBA significantly blocked the bell-shaped effects of ManN on BCEC proliferation. Pre-treatment of cells with 1 mM 4-PBA for 8 hours abrogated additive effects of 40 μM ManN and 5 ng/ml VEGF and protected cells from toxic effect induced by 2 mM ManN. n=3 independent samples were used. For each study, a representative experiment is shown from 2-3 independent studies. Data are means+/−SD, Asterisks indicate a significant difference compared with control. Statistical analysis was done by 2-tailed, two-sample unequal variance t test. *p<0.05, **p<0.01.

FIGS. 6A to 6J are charts showing effects of ManN on non-endothelial cells of bovine, mouse or human origin. ManN did not promote growth of Calu6 (FIG. 6A), A673 (FIG. 6B), U87MG (FIG. 6C) and 4T1 (FIG. 6D) tumor cells. 10% FBS was used as positive control for Calu6 and A673, whereas 10 ng/ml bFGF and 1 μg/ml human apo-transferrin were used as positive controls for U87MG and 4T1, respectively. Similarly, no increases in proliferation were induced by ManN on AML12 (FIG. 6E), bovine pituitary cells (FIG. 6F), NIH3T3 cells (FIG. 6G), human RPEs (FIG. 6H), human dermal fibroblasts (FIG. 6I), and human keratinocytes (FIG. 6J), alone or in combination with growth factors. Proliferation quantification was performed using AlamarBlue or MTS (for 4T1 cells). n=3 independent samples were used. Inserted in the charts in FIGS. 6A to 6J are representative western blot analyses showing dose-dependent effects of ManN and mannose at 400 μM (2,4) and 2 mM (3,5) on bFGFR1 or (31 integrin (for 4T1, AML12, NIH3T3 cells, human skeletal muscle cells, human dermal fibroblasts and human keratinocytes) molecular mass compared to the untreated control (1). β-actin served as loading control. GM: Growth Media. Proteins were separated on NuPAGE 3-8% Tris-Acetate gel for western blot analysis. For each study, a representative experiment is shown from 2 independent studies. Asterisks indicate a significant difference compared with control. When statistical analysis was done using a different control, bracket was used between specified groups. Data were means+/−SD of the mean or an average when n=2. Statistical analysis was done by 2-tailed, two-sample unequal variance t test. *p<0.05, **p<0.01.

FIGS. 7A to 7F represent examples showing effects of protein glycosylation inhibitors on BCEC proliferation. FIG. 7A includes images of samples showing dose-dependent stimulation of BCEC proliferation by various inhibitors of glycosylation. Inhibitors were added at concentrations ranging from 0.01 to 100 μM for 3 days, with or without 5 ng/ml VEGF. At the end of the experiment, cells were fixed and stained with crystal violet. A representative experiment is shown. Kifunesine (Kif), an ERα-1,2-mannosidase I and Golgi α-mannosidase I inhibitor; Castanospermine (Cas), an a-glucosidase inhibitor. Cell-covered areas in various treatment groups were quantified by ImageJ software. FIG. 7B is a chart showing dose-dependent effects of Kif and Cas in promoting BCEC proliferation with or without 5 ng/ml VEGF. n=3 independent samples were used. FIG. 7C includes western blot images showing that both inhibitors reduced VEGFR2 molecular mass and induced Bip expression in a dose-dependent fashion as assessed by western blot analysis. Proteins from total cell lysates were separated using 3-8% Tris-Acetate gel. BCECs were treated with various inhibitors for 24 hours. Quantification of western blots was done by densitometry. (3-actin was the loading control. FIG. 7D is a chart showing acceleration of closure of monolayer gaps by Kif and Cas in BCEC scratch assay, with controls for Kif (H₂O) and Cas (DMSO). Gaps were quantified using AxioVision LE Rel.4.4 software. n=3 independent samples were used. Scale bar=400 μm. FIG. 7E is a western blot image showing activation of AKT and JNK in BCECs by glycosylation inhibitors at 40 μM and VEGF at 10 ng/ml. However, Cas did not activate ERK. Quantification of phosphorylated AKT, JNK and ERK was done by densitometry analysis relative to total protein. FIG. 7F is a chart showing pre-treatment of BCECs with 5 μM SP600125 for 2 hours significantly blocked the effects of both glycosylation inhibitors on BCEC proliferation. n=3 independent samples were used. A representative experiment is shown from 2-4 independent studies. Data shown are means+/−SD. Statistical analysis was done by 2-tailed, two-sample unequal variance t test. *p<0.05, **p<0.01.

FIGS. 8A to 8D represent examples showing topical application of ManN and VEGF stimulated angiogenesis and accelerates wound healing in mice. FIG. 8A is a chart showing effect of ManN on wounds. Wounds were made on the dorsal skin of mice by 6 mm punch. VEGF and ManN each was administered daily at 20 μg per wound in 25 μl PBS for the first 4 days, with PBS as control. A 10-day wound healing study with 5 mice in each group. Wound closure rate (%) was quantified by Image J software in two independent studies. Asterisks indicated a significant difference compared with the control at each time point. FIG. 8B includes images resulting from a 4-day wound healing study with images of the wound healing process at day 1, day 2 and day 4. n=5 animals/treatment group were used. FIG. 8c includes representative images of immunohistochemical staining for CD31 in PBS control group and in VEGF and ManN combination groups (scale bar=200 μm). FIG. 8D is a chart showing quantification of CD31-positive blood vessel (red dotted circles) density around the wound areas was counted by eyes under microscopy (20× magnification). Data are means+/−SD. Statistical significance was further confirmed using Wilcoxon rank-sum test between treatment groups of interest. Asterisks indicated a significant difference compared with the PBS control. For each study, a representative experiment is shown. n=3 animals/treatment group were used. When statistical analysis was done using a different control, a line was used between specific groups. Statistical analysis was done by 2-tailed, two-sample unequal variance t test. *p<0.05, **p<0.01.

FIGS. 9A to 9D represent examples showing ManN accelerates blood perfusion recovery in a mouse ischemic hindlimb model. FIG. 9A includes images resulting from serial laser Doppler analysis of blood perfusion in hindlimbs of ManN-treated, Kif-treated and control mice. Different colors were used to indicate blood perfusion in the ischemic limb (ligated; left side) to nonischemic limb (sham; right side). Representative images at week 0 and week 1 are shown. FIG. 9B is a chart showing quantification of blood perfusion ratio between region 2 (ischemic; left limb) and region 1 (nonischemic; right limb), n=8 animals/treatment group. FIG. 9C includes images of sample tissue. Three weeks after surgery, skeletal muscle tissues were harvested and fixed. CD31 immunostaining on these tissue sections was performed to label the vasculature. H&E staining was also performed. Representative CD31 staining and H&E histological image of ischemic hindlimbs 21 days after surgery were shown. Scale bar=50 μm. FIG. 9D is a chart showing quantification of vascular density by CD31 immunostaining performed using ImageJ software, n=8 animals/treatment group, 3 independent experiments; Data are means+/−SEM. Statistical analysis was done by 2-tailed, two-sample unequal variance t test. *p<0.05, **p<0.01.

FIGS. 10A and 10B represent examples showing ManN promotes retinal neovascularization in mice. FIG. 10A includes images of tissue. Intravitreal injection of ManN increases blood vessel density. Adult mice were intravitreally injected once 500 ng of ManN, Kif or 200 ng of bFGF. PBS was used as vehicle control. Seven days after injection, PFA-fixed retinas were subjected to CD31 immunofluorescence. Representative images of CD31-positive vessels are shown. n=10 animals/treatment group, 3 independent experiments, scale bar=50 μm FIG. 10B is a chart showing vascular density determined with ImageJ software, n=10 animals/treatment group, 3 independent experiments. Data were means+/−SEM. Statistical analysis was done by 2-tailed, two-sample unequal variance t test. *p<0.05, **p <0.01, ***p<0.001.

FIGS. 11A to 11D represent examples showing that ManN, but not structurally related molecules, stimulates endothelial cell proliferation. FIG. 11A is a chart showing additive effects of ManN and bFGF on BCEC proliferation. Bell-shaped effects of ManN on BCEC proliferation. BCECs were treated with ManN ranging from 0.4-400 μM for 5-6 days, with or without 20 ng/ml bFGF. At the end of the experiment, proliferation was quantified using AlamarBlue. FIG. 11B is a chart showing that additive effects of VEGF and ManN on BCEC proliferation are dependent on glycolysis pathway. Proliferation assays were carried out in low glucose DMEM media without growth factors or in DMEM media without glucose and pyruvate. Asterisks indicate a significant difference compared with no treatment control. Statistical analysis was also done to compare VEGF alone and VEGF plus ManN treatment groups for cells grown in two different assay media. FIGS. 11C and 11D are charts showing the effect on BCECs of various agents at 0.04 μM-5 mM in the absence (FIG. 11C) or presence (FIG. 11D) of 5 ng/ml VEGF. n=3 independent samples were used. For each study, a representative experiment is shown from 2 independent studies. Data are means+/−SD. Statistical analysis was done by 2-tailed, two-sample unequal variance t test. *p<0.05, ** p<0.01.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the present disclosure. Although any methods and materials similar or equivalent to those described herein can be used in the practice of the present disclosure, the exemplary methods, devices, and materials are described herein.

The present disclosure provides pharmaceutical compositions and methods for treating an ischemic condition in a subject, including administering to the subject in need thereof an effective amount of hexosamine D-mannosamine (ManN). Because ManN is converted to ManN-6-phosphate (ManN-6p) in vivo, the present disclosure provides for the use of such metabolic precursors and derivatives.

ManN is a hexosamine with an ability to inhibit protein post translational modifications, activate stress pathways and show additivity with VEGF in promoting endothelia; cell (EC) proliferation and angiogenesis. Effects of ManN on ECs and angiogenesis have not been previously reported. Without being bound by theory, using well-known glycosylation inhibitors together with ManN may result in a link between changes in glycosylation patterns in mammalian ECs and angiogenesis. The effects of ManN on endothelial cells may be independent of VEGFR2 activation.

ManN was discovered in the 1960s as a bacterial wall component [15], and accounts for 5-10% of capsular polysaccharides [45]. The related N-acetyl mannosamine is thought to be an intermediate in the biosynthesis of sialic acids [46]. Over the years, multiple effects of ManN on enzymes, growth factor-mediated signaling, protein stability and cell viability were documented [47-50]. Most of these effects were not unique to ManN and could be elicited by other hexosamines. In addition, they required high concentrations [45, 48]. ManN was reported to have antitumor properties [47], to stimulate osteogenic differentiation [48, 51] and to protect articular cartilage [49]. More recently, ManN was used as an intermediate in modifying various molecules/nanoparticles and in the synthesis of non-natural ManNAc analogs for the expression of thiols on cell-surface sialic acids to facilitate high-throughput screening [53]. However, to date no effects of ManN on ECs have been described.

ManN had been previously reported to affect formation of lipid-linked oligosaccharides (LLO) in MDCK cells possibly by inhibiting the a-1,2-mannosyl transferases[24]. Upon ManN treatment, major oligosaccharides associated with the dolichol were Man5GlcNAc2 and Man6GlcNAc2 rather than Glc3Man9GlcNAc2 which was normally found in MDCK cells. In addition, ManN was reported to change protein GPI biosynthesis and hybrid glycans production in the ER [54-56]. However, none of the angiogenic-related proteins previously examined are GPI-anchored. Without being bound by theory, a decrease in Man-9 could be a direct result of inhibiting LLO donor synthesis, i.e. Glc3Man9GlcNAc20PP-Dol formation, which is then transferred from the dolichol donor onto the polypeptide. In embodiments, Man-5 may be significantly increased over 24 hours by treating cells with 40 μM ManN. ManN may be not affect α-mannosidases in the ER.

Activation of PI3K-AKT, PLCγ-ERK and p38 is associated with VEGFR2-mediated EC survival, proliferation and migration. Other cellular metabolic stress sensors, such as AMPK (AMP-activated protein kinase), could also confer stress adaptation and promote EC survival via eNOS [57]. Without being bound by theory, ERK, AKT, mTOR, AMPKα, eNOS, and ACC activation is a general phenomenon for hexosamines and mannose. However, activation of the JNK/c-Jun and UPR pathways in BCECs is unique to ManN as well as the glycosylation inhibitors. Glycosylation is required for correct protein folding in the ER [26]. A link between LLO inhibition and activation of UPR has been reported [58]. In fact, notwithstanding the complexity of ManN actions, LLO inhibition, followed by UPR activation, seems a plausible explanation for the reported ManN effects.

ECs are able to cope with acute/minor ER stress resulting from glycosylation inhibition by activating the UPR pathway. UPR detects misfolded proteins accumulated in the ER and initiates a response to maintain cellular homeostasis via induction of Bip, a major ER chaperon protein [29]. BiP binds to hydrophobic patches exposed on nascent or incompletely folded proteins that are often non-glycosylated. ManN exhibits a strong induction of Bip expression relative to hexosamines. Similar effects on stress pathway activation may result from glycosylation inhibitors Kif and Cas.

Glycosylation inhibition is thought to be a new pharmacological strategy targeting metabolic pathways essential for excessive angiogenesis in various pathological conditions, and glycosylation inhibitors are expected to have anti-angiogenic and anti-metastatic properties [10, 59, 60]. Glycosylation has been shown to be involved in cellular stress response and compensatory angiogenesis in response to VEGF-VEGFR2 signaling blockade [61]. Stress-induced O-GlcNAcylation was previously reported to promote survival in response to DNA damage, ER stress, glucose deprivation and hypoxia in a variety of cell types [62]. Without being bound by theory, glycosylation inhibition may be linked to angiogenesis promotion, and inhibiting glycosylation within the tumor microenvironment may result in stimulation rather than suppression of tumor angiogenesis.

In embodiments, ManN may be used to promote angiogenesis in a mouse skin injury model, accompanied by accelerated wound closure. In embodiments, ManN may be used to stimulate angiogenesis and blood flow recovery in ischemic hindlimbs of mice. Combinations of ManN, or other glycosylation inhibitors, with VEGF-A, may have advantages over monotherapy for the treatment of ischemic disorders. A lack of direct permeability-enhancing effects of ManN may result in less edematous tissues. In this context, damage to lung endothelium is a central pathogenic event in the respiratory failure associated with a variety of infections, including SARS-CoV-2 [65]. An endothelial cell mitogen like ManN, devoid of permeabilizing effects, may help protect and stabilize blood vessels and thus limit tissue damage.

In embodiments, intravitreal administration of ManN may be used to enhance retinal neovascularization, for example, in therapeutic applications in ocular diseases. 10-15% of patients with intermediate AMD progress to the neovascular form, while the remaining patients may develop geographic atrophy (GA) [1]. Previous studies have shown that loss of choroid capillaries is frequently detected in GA, which raises the possibility that regeneration/protection of choroid capillaries may be a strategy for GA treatment [66].

In embodiments, the administration is effective to promote endothelial cell proliferation and angiogenesis in the subject. In embodiments, the method further includes administering to the subject in need thereof an effective amount of an N-glycosylation inhibitor. In embodiments, the method further includes administering to the subject in need thereof an effective amount of VEGF.

In embodiments, the ischemic condition is caused by a disease or a trauma. The present disclosure provides for treatment of a number of conditions characterized by reduced perfusion, including but not limited to diabetic ulcers, macular degeneration, peripheral arterial disease (PAD), limb ischemia, brain or cerebral ischemia, and coronary ischemia.

In embodiments, the administration is intravenous, intraperitoneal, or intravitreal.

In embodiments, the present disclosure provides pharmaceutical compositions and methods for inducing angiogenesis in a subject, including administering to the subject in need thereof an effective amount of hexosamine D-mannosamine (ManN).

In embodiments, the administration is effective to reduce ischemia in the subject. In embodiments, the ischemia may include brain ischemia. The administration may be effective in preventing, reducing, or treating conditions associated with brain ischemia, such as edema, ischemic stroke, or infarctions. In embodiments, the method further includes administering to the subject in need thereof an effective amount of an N-glycosylation inhibitor. In embodiments, the method further includes administering to the subject in need thereof an effective amount of VEGF.

In embodiments, the subject is in need of inducing angiogenesis due to an ischemic condition is caused by a disease or a trauma.

In embodiments, the administration is intravenous, intraperitoneal, or intravitreal.

In embodiments, the present disclosure provides pharmaceutical compositions and methods for inhibiting protein glycosylation in a cell, including administering to the cell an effective amount of hexosamine D-mannosamine (ManN).

In embodiments, the administration is in vivo. In embodiments, the administration is ex vivo. In embodiments, the administration is effective to stimulate EC proliferation and angiogenesis. In embodiments, the administration is effective to activate JNK and an unfolded protein response caused by ER stress.

In embodiments, the administration is effective to induce changes in N-glycan and O-glycan profiles. In embodiment, the administration is effective to induce reduction in Man6GlcNAc2 (Man-6), Man-8 and Man-9 in total oligomannose N-glycan content compared to an untreated control, accumulation of Man-5 and Man-7, and to decrease O-glycosylation following treatment with ManN.

Conversely, in embodiments the present disclosure provides pharmaceutical compositions and methods for inhibiting angiogenesis, including but not limited to methods for treating malignant tumors and intraocular neovascular disorders in a subject, including administering to the subject in need thereof an effective amount of an inhibitor of hexosamine D-mannosamine (ManN) or reducing the amount of ManN available to the subject.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method.

It is understood that aspects and embodiments of the present disclosure described herein include “consisting” and/or “consisting essentially of” aspects and embodiments. As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a pharmaceutical composition, and/or method that includes materials, steps, features, components, or elements, in addition to those literally disclosed, provided that these additional materials, steps, features, components, or elements do not materially affect the basic and novel characteristic(s) of the claimed subject matter. The term “consisting essentially of” occupies a middle ground between “comprising” and “consisting of”.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or other mammalian subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety. The term “derivative” refers to a chemical substance related structurally to another substance, or a chemical substance that can be made from another substance (i.e., the substance it is derived from), e.g., through chemical or enzymatic modification.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

### EXAMPLES

As disclosed in the following examples, hexosamine mannosamine (2-Amino-2-deoxy-D-mannose or ManN hereafter) inhibits protein glycosylation and yet stimulates EC proliferation in vitro. The biological effects of ManN in other in vitro and in vivo models, as well as its possible mechanisms of action were investigated. ManN is an EC mitogen and survival factor for bovine and human microvascular EC, with an additivity with VEGF. ManN inhibits glycosylation in ECs and induces significant changes in N-glycan and O-glycan profiles. ManN and two N-glycosylation inhibitors stimulate EC proliferation via both JNK activation and the unfolded protein response caused by ER stress. ManN results in enhanced angiogenesis in a mouse skin injury model. ManN also promotes angiogenesis in a mouse hindlimb ischemia model, with accelerated limb blood flow recovery compared to controls. In addition, intraocular injection of ManN induces retinal neovascularization. Therefore, activation of stress pathways following inhibition of protein glycosylation can promote EC proliferation and angiogenesis and may represent a therapeutic strategy for treatment of ischemic disorders.

### Example 1

The effect of ManN on EC proliferation was assessed. A library of 619 highly purified metabolites encompassing a broad spectrum of chemical entities was screened for their ability to affect growth of bovine choroidal microvascular EC (BCEC), in the presence or in the absence of VEGF. This and similar assays have been previously employed to identify and characterize angiogenesis stimulators and inhibitors [12-14]. Under the conditions tested, little or no proliferation was detected in the absence of VEGF.

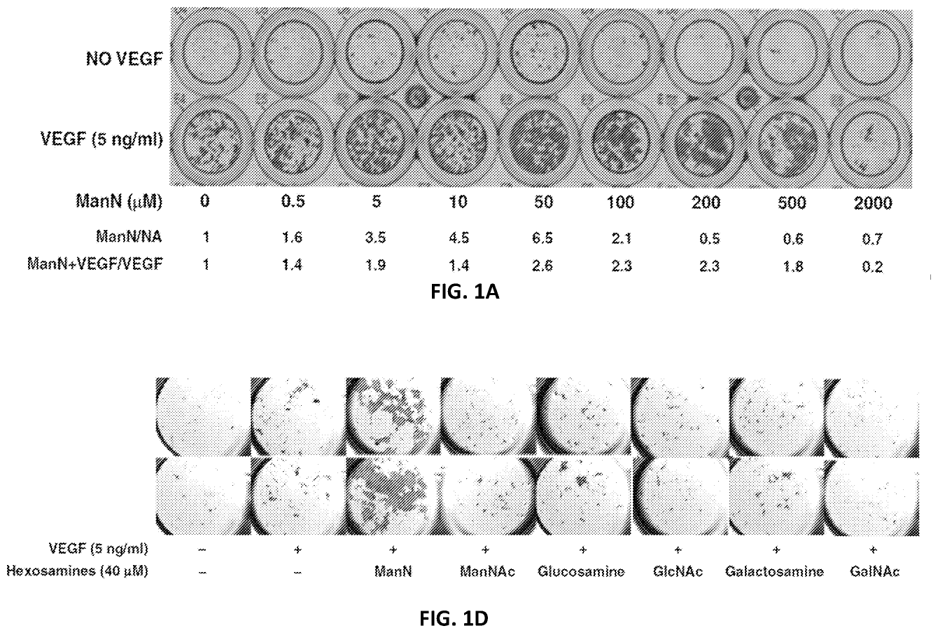

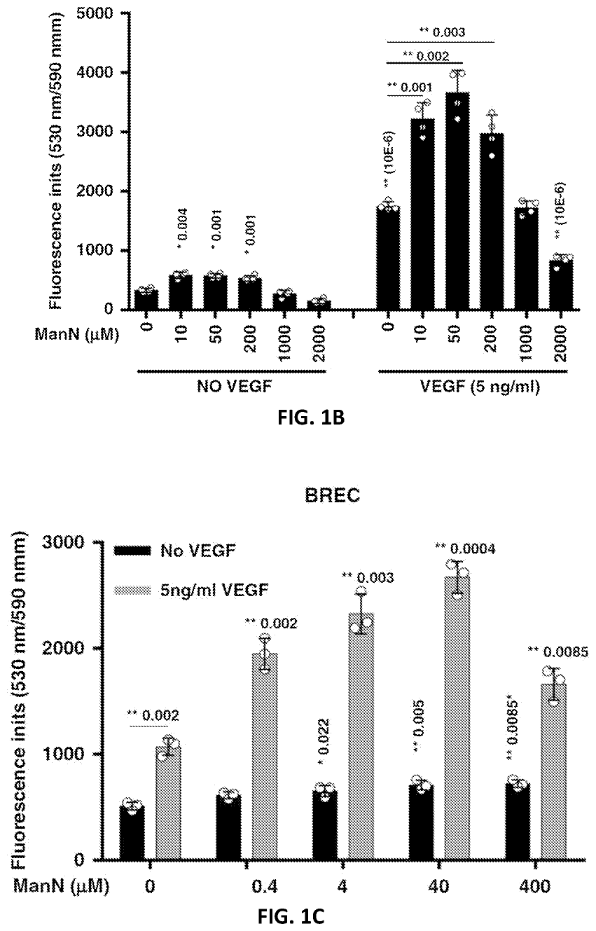

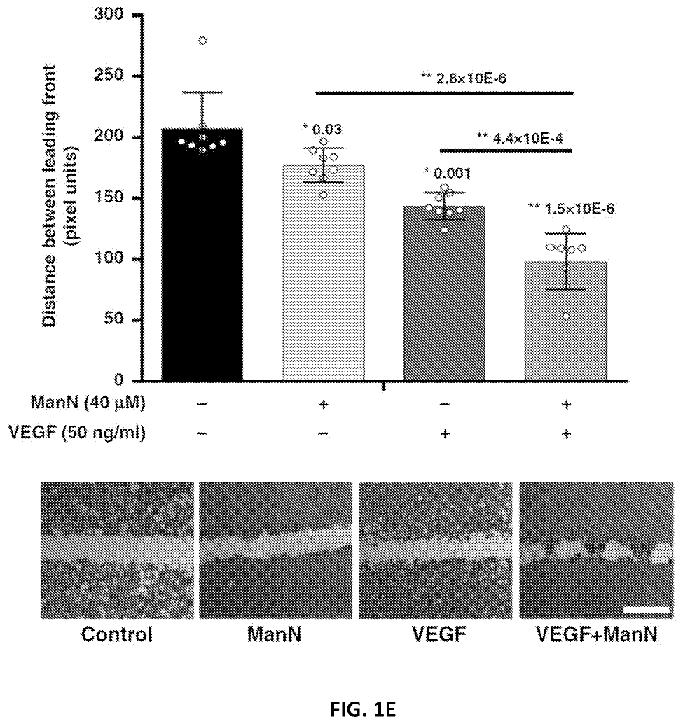

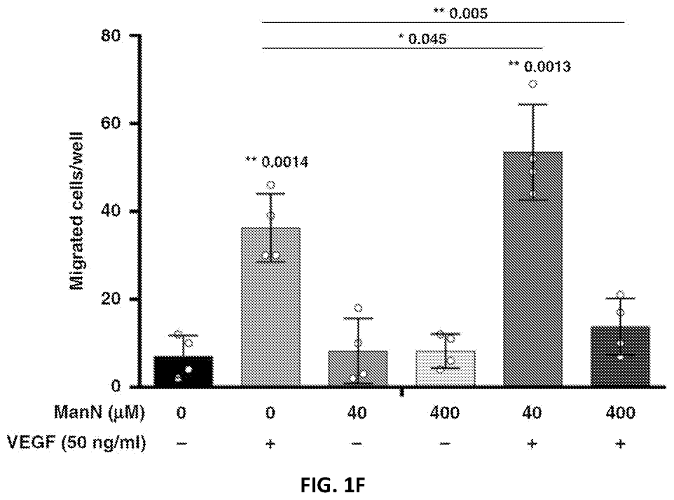

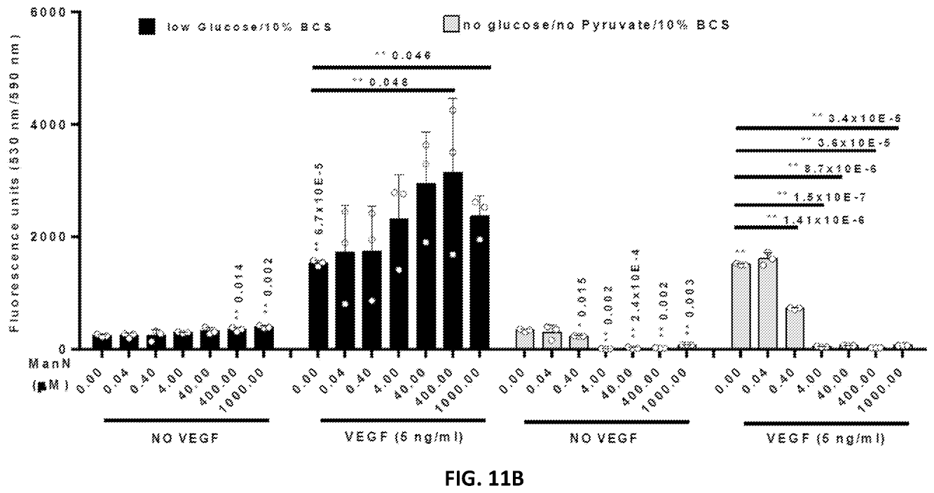

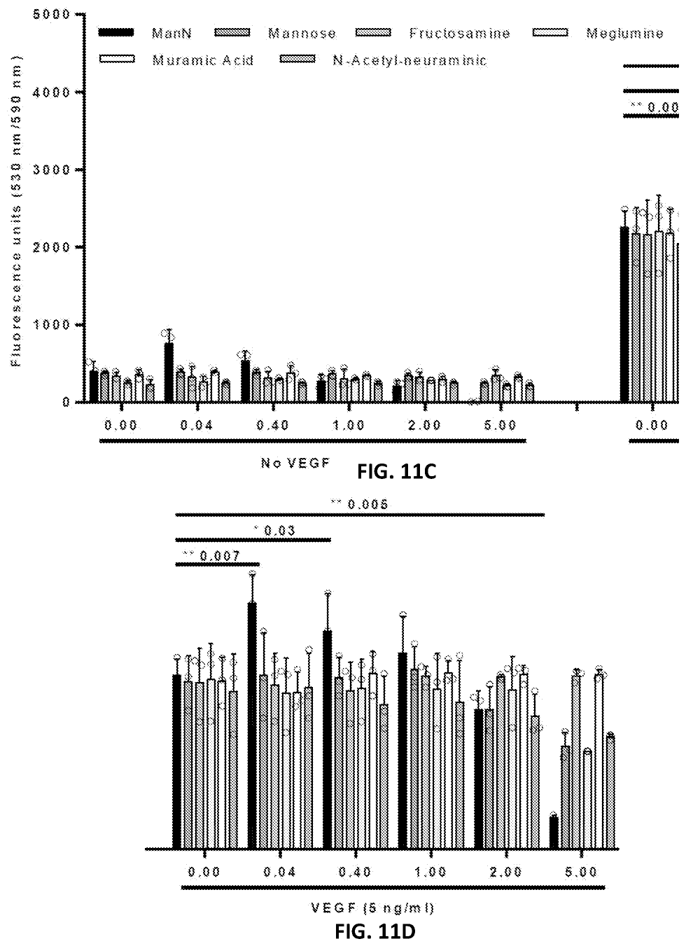

An initial screening was done, testing each compound at the concentrations of approximately 1 and 10 uM (assuming a molecular mass of 100 Da for each compound), with or without 5 ng/ml VEGF, which could induce ˜4-5 fold increase in cell proliferation. Six compounds of various chemical nature showed some inhibitory or stimulatory activity. The analysis was focused on one of these, ManN, a hexosamine originally identified as a component of bacterial cell wall because it showed the most potent and consistent effects. ManN had significant stimulatory effects in the 5-500 uM dose range and was also additive with VEGF in promoting BCEC proliferation. Dose-dependent effects of ManN on BCEC proliferation, in the absence or in the presence of VEGF, are shown in FIGS. 1A and 1B. A maximal ˜6.5 fold increase in EC-covered surface by ManN at 50 uM alone (FIG. 1A) or ˜2.5-3 fold increase in fluorescence units upon AlamarBlue addition (FIG. 1B) was obtained when cells were treated with 50 uM ManN and 5 ng/ml VEGF, compared to VEGF alone. AlamarBlue detects mitochondria activity as an indication of cell viability which correlates with cell number at certain ranges [16]. The effects of ManN had a bell-shaped dose-response curve, with inhibition at higher concentrations (FIGS. 1A and 1B). Additive effects of ManN in promoting BCEC proliferation were observed also with bFGF (FIG. 11A) and in bovine retinal EC (BRECs) (FIGS. 11C and 11D).

Various hexosamines (galactosamine, glucosamine and their N-acetyl derivatives) were tested alongside ManN in the BCEC proliferation assay. However, none of these hexosamines had significant stimulatory effects (FIG. 1D). Several structurally related molecules such as D-isoglucosamine (fructosamine), meglumine, muramic acid, N-Acetyl-neuraminic acid (sialic acid present in all mammalian cells), glucose, and mannose were also tested. None of these molecules stimulated BCEC proliferation, with or without VEGF (FIGS. 11C and D).

ManN entered and accumulated inside the cells in a concentration-dependent manner. 0.66 nmol of ManN were detected in 1 mg cell lysate when BCECs were treated with 400 uM ManN for 2 hours. Following entry into the cells, ManN, but not mannose, is quickly converted to ManN-6-phosphate (ManN-6p) [17]. No incorporation of ManN was detected in N-glycans. Efficient uptake of ManNAc, and mannose has been reported [18, 19].

The effects of ManN on BCEC proliferation were dependent on cellular glycolysis. The additivity between ManN and VEGF was abolished when glucose-free media was used. On the other hand, the activity of VEGF was not dependent on the glycolysis pathway (FIG. 11B). However, there was a significant cytotoxicity with as little as 4 uM ManN, even in the presence of VEGF, in glucose-free media.

To further characterize the effects of ManN on EC survival, proliferation and migration, confluent BCEC monolayers were mechanically wounded. FIG. 1E shows that 40 uM ManN or 50 ng/ml of VEGF significantly accelerated BCEC migration and/or proliferation as reflected by more complete closure of the “scratched” area, compared to the control group after 48 hours. Similar to the proliferation assays, additivity was observed when cells were treated with both ManN and VEGF (FIG. 1E). In addition, ManN at 40 uM showed a significant additivity with VEGF in promoting BCEC migration (FIG. 1F).

The observations were extended to human retinal microvascular EC (hRMECs), HUVEC and dermal microvascular endothelial cells (hDMVECs). ManN by itself stimulated HUVEC and hDMVECs growth. In addition, there was a dose-dependent additivity with VEGF in all EC types tested, with minimal toxicity even at 5 mM. Likewise, stimulation of migration and wound closure was observed in HUVEC treated with 40 uM ManN alone (migration assay) and/or in combination with 50 ng/ml VEGF (scratch assay).

### Example 2

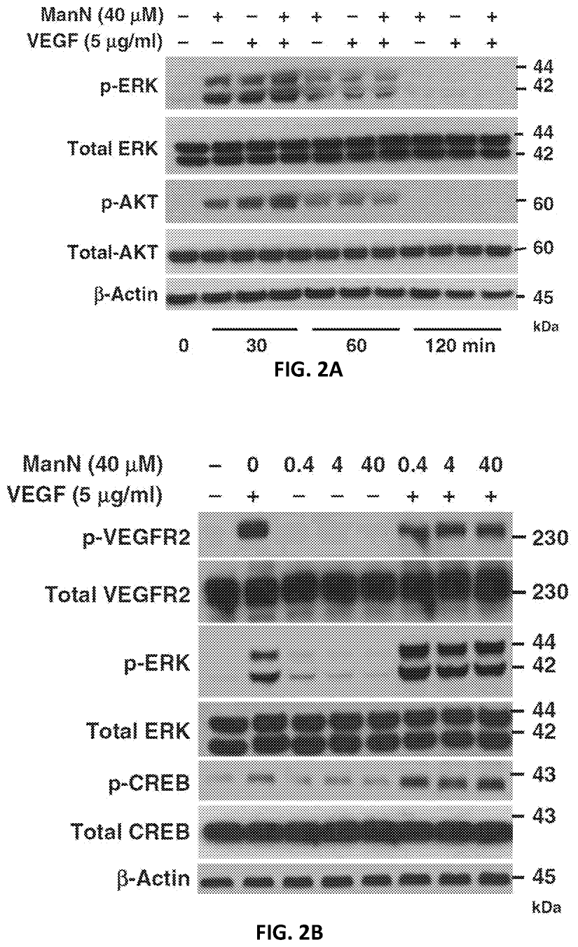

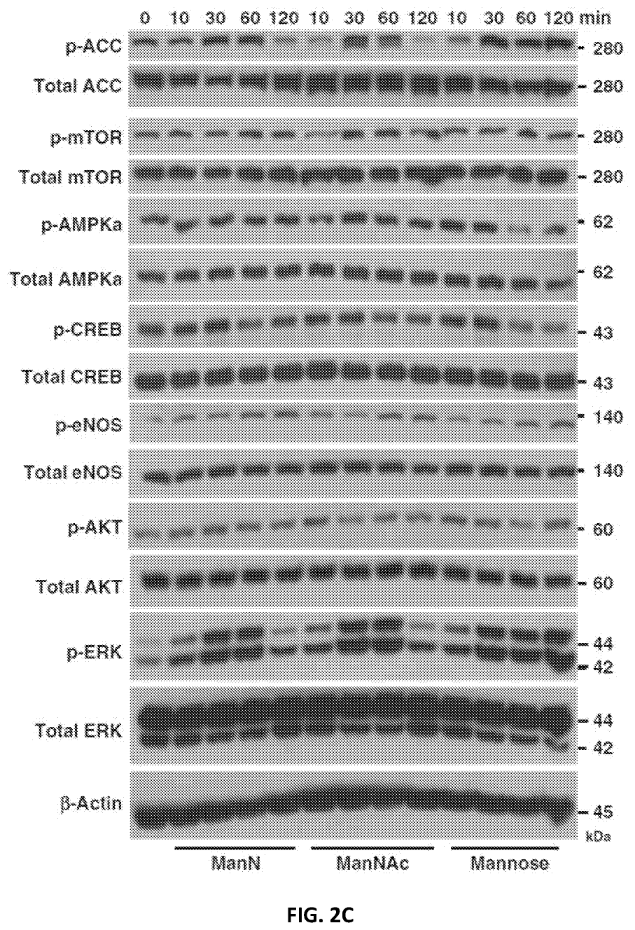

Activation of ERK, AKT, mTOR, CREB, AMPK, ACC and eNOS is not unique to ManN. It has been reported that cross-talk between signaling and metabolic pathways in the vasculature, such as insulin signaling and glucose metabolism in ECs, involves AKT and STAT3 activation. Together, they affect glycolysis, EC sprouting, proliferation and migration [20]. The effect of ManN and/or VEGF on activating major signal transduction pathways known to promote proliferation, such as ERK, AKT, mTOR and CREB (cAMP response element binding protein) in BCECs was assessed. ManN activated ERK, AKT, mTOR and CREB at 40 uM. Stimulation of ERK, AKT and CREB was rapid and occurred within 10-30 minutes after adding ManN (FIGS. 2A to 2C). Further, an enhancement in activation of ERK, AKT and CREB was observed when both ManN and VEGF were present compared to ManN or VEGF alone (FIGS. 2A and 2B). The effect of ManN on activating the ACC (Acetyl-CoA carboxylase)/eNOS (endothelial nitric oxide synthase 3) pathway was assessed. Activation of the energy sensor AMPK (AMP-activated protein kinase) leads to eNOS activation and NO (nitric oxide) production; the latter exerts bell-shaped effects on EC proliferation [21]. Both eNOS and ACC were significantly activated by 40 uM ManN within 10-30 minutes (FIG. 2D). However, activation of ERK, AKT, mTOR, CREB, ACC and eNOS was not unique to ManN. Indeed, other hexosamines such as ManNAc and mannose induced a similar activation of these signal transduction pathways (FIG. 2C). While activation of these common proliferation pathways likely contributed, without being bound by theory, some unique mechanism(s) may be implicated in the EC mitogenic effects of ManN.

### Example 3

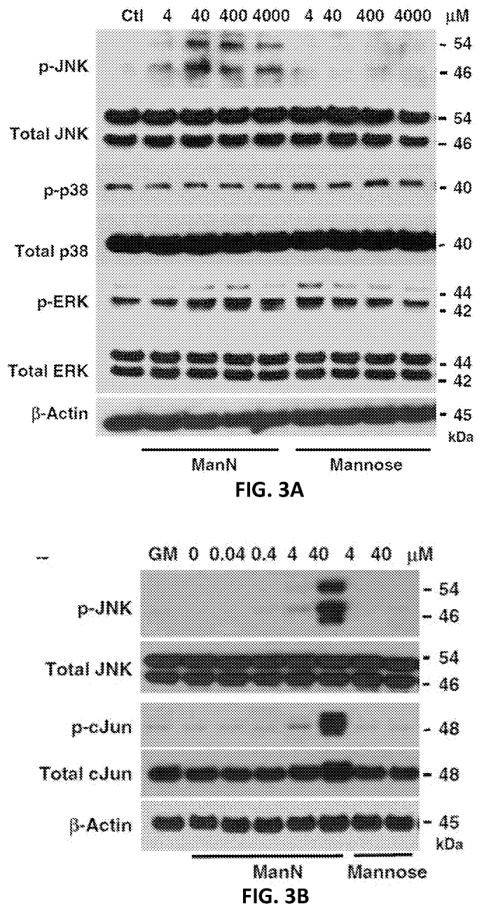

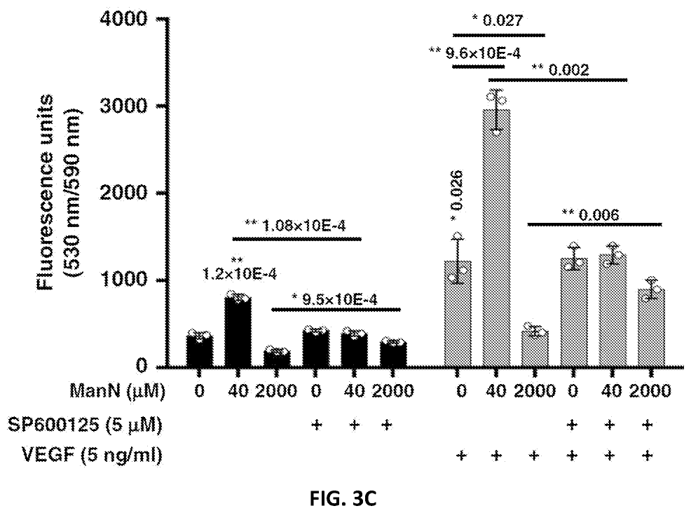

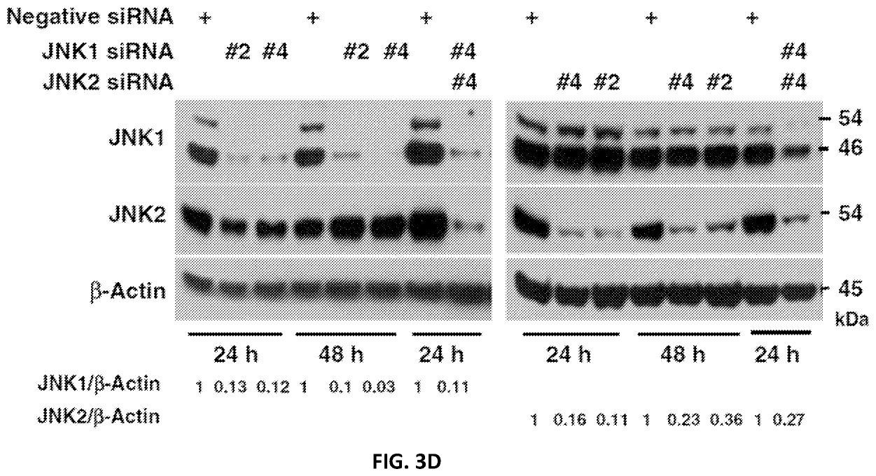

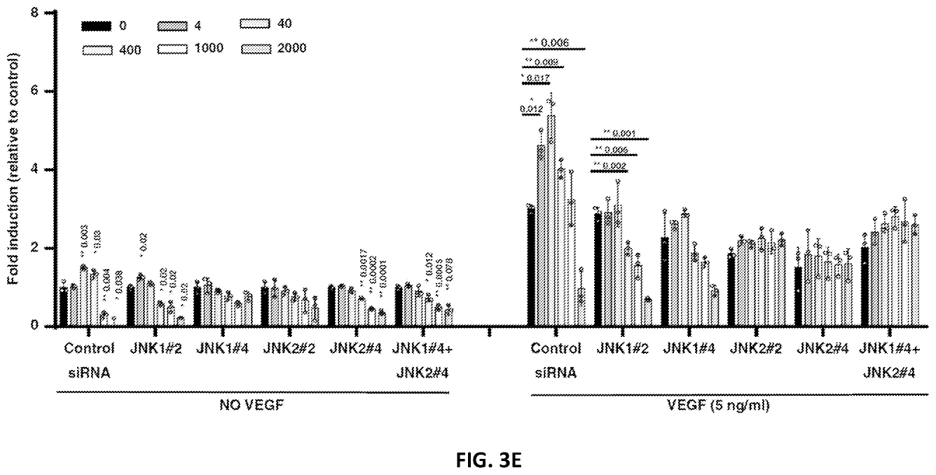

A series of specific pharmacological inhibitors was used to identify JNK/c-jun as a signal transduction pathway uniquely activated by ManN, among hexosamines. Western blot analysis revealed that, among three MAPK family members (ERK, p38, JNK), JNK was specifically activated by ManN. When growing BCECs were switched to proliferation assay media without growth factors, JNK and its downstream c-Jun were significantly activated by ManN in a dose-dependent manner, but not by mannose (FIGS. 3A and 3B). ManN, but not other hexosamines tested, activated the JNK pathway. Treatment of BCECs with the JNK specific inhibitor SP600125 (5 uM) abolished the effects of ManN on BCEC proliferation (FIG. 3C).

The effects of ManN on BCECs were assessed following transfection with siRNA against JNKs (namely JNK1 and JNK2, as JNK3 is not expressed in BCECs). Knocking down ˜80% of either JNK1 and/or JNK2 by two independent siRNAs against JNK1 or JNK2 abolished mitogenic effects of ManN on BCECs at uM concentrations (FIGS. 3D and 3E), indicating that both JNK1 and JNK2 are important in transducing stress signals.

### Example 4

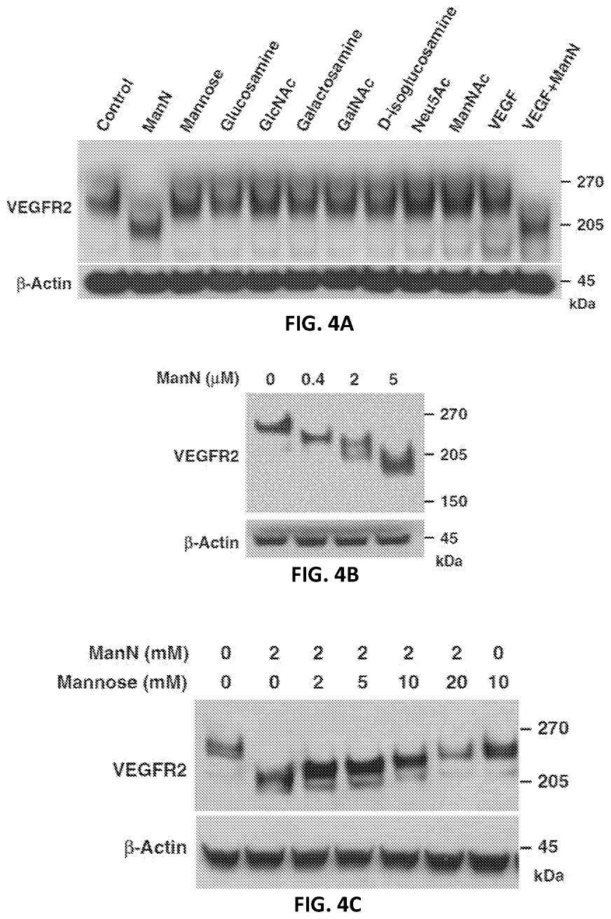

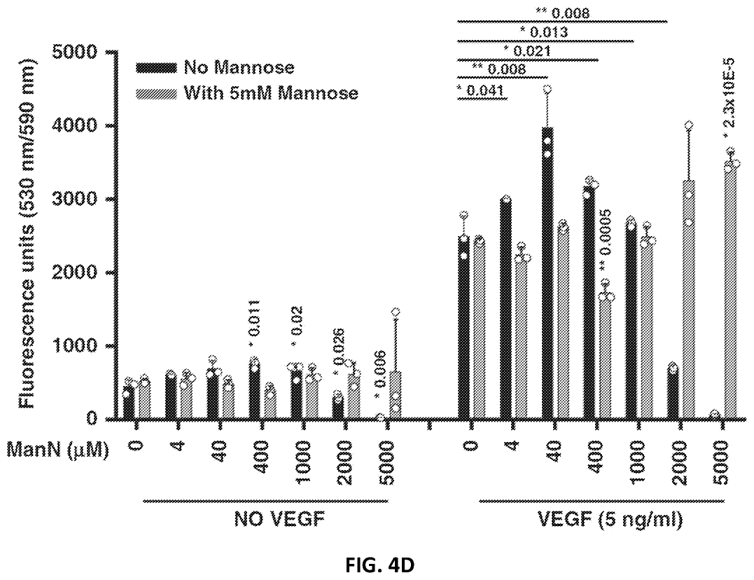

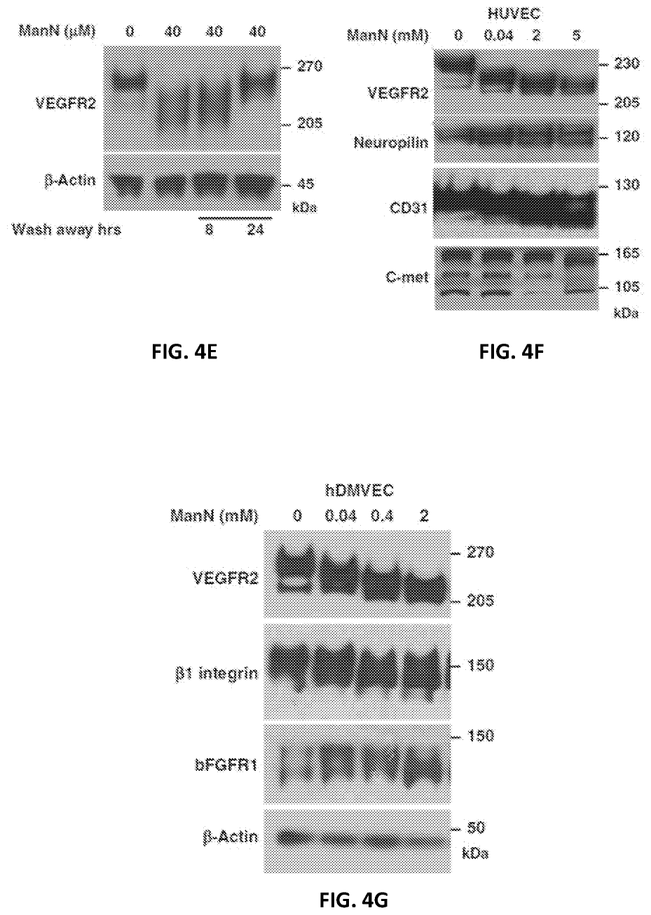

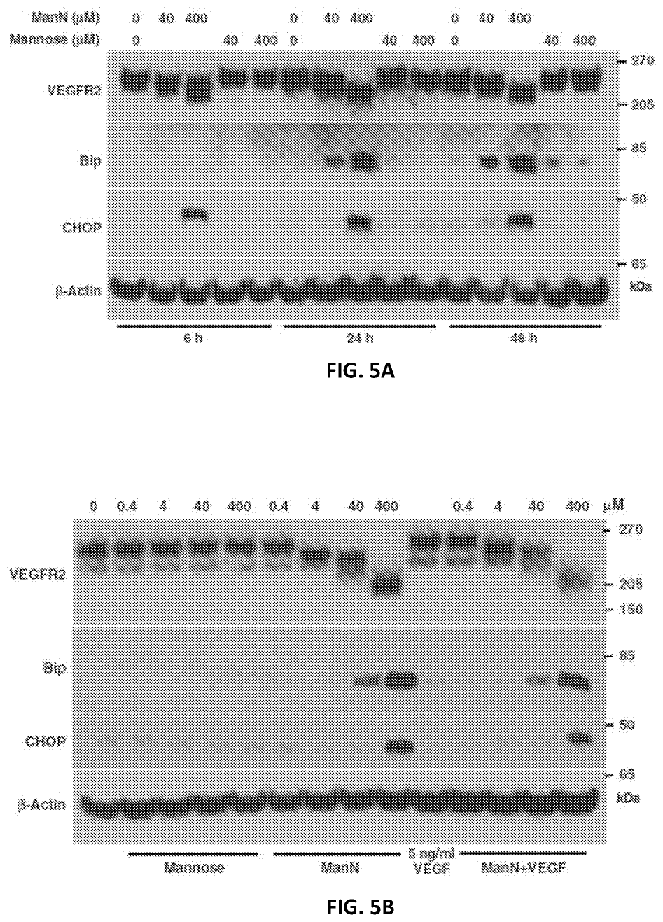

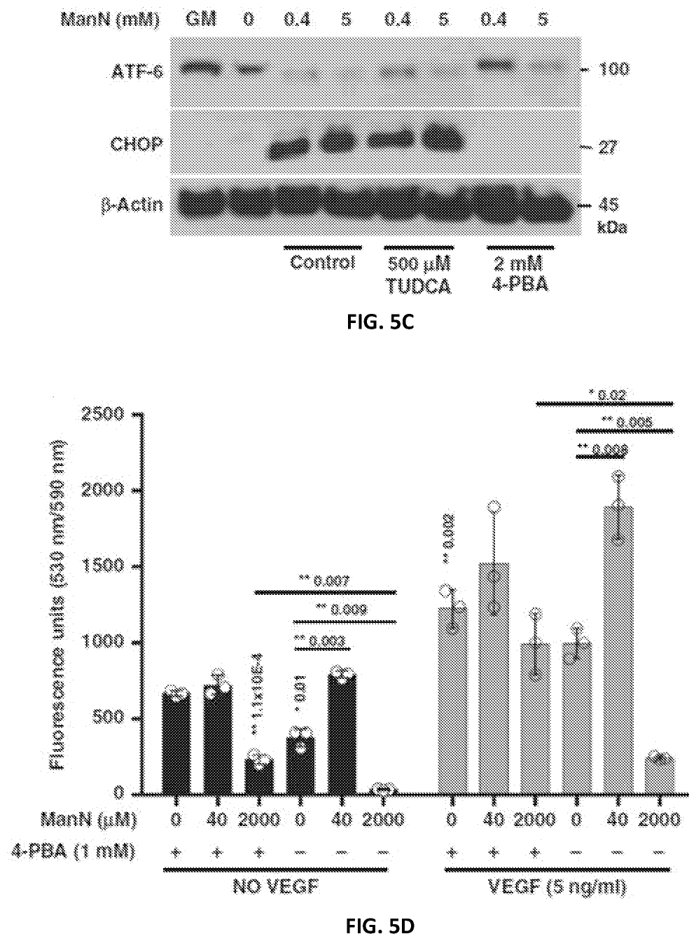

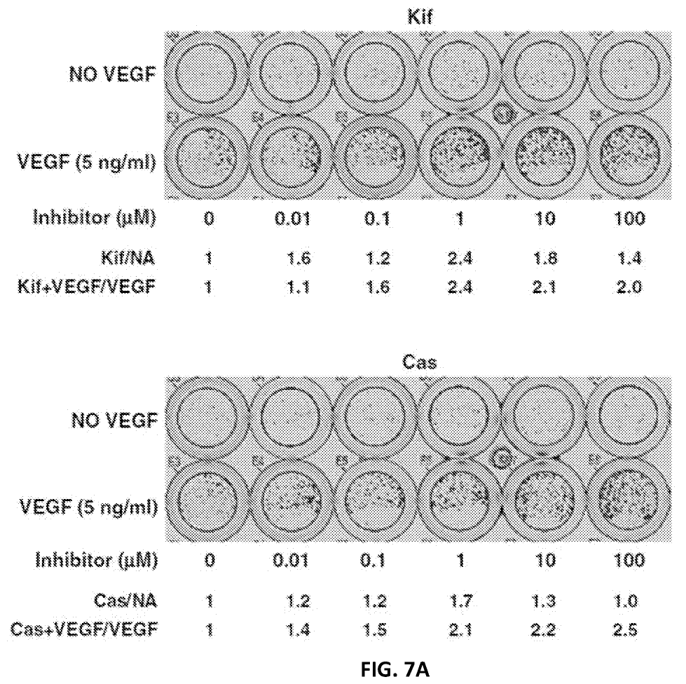

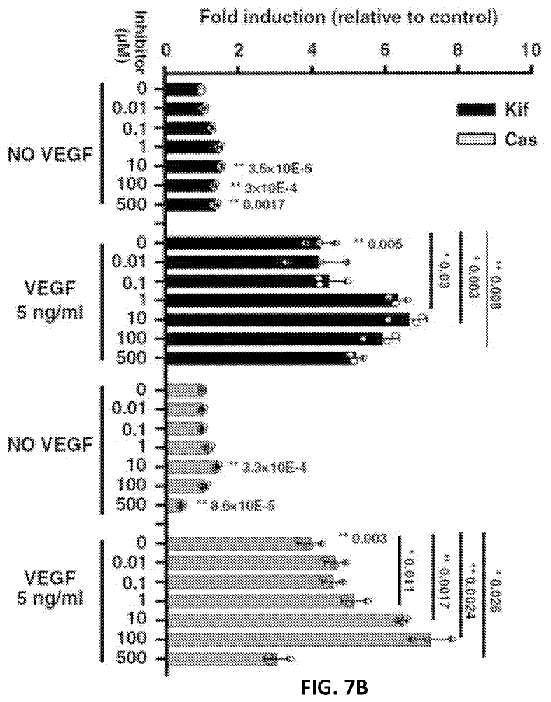

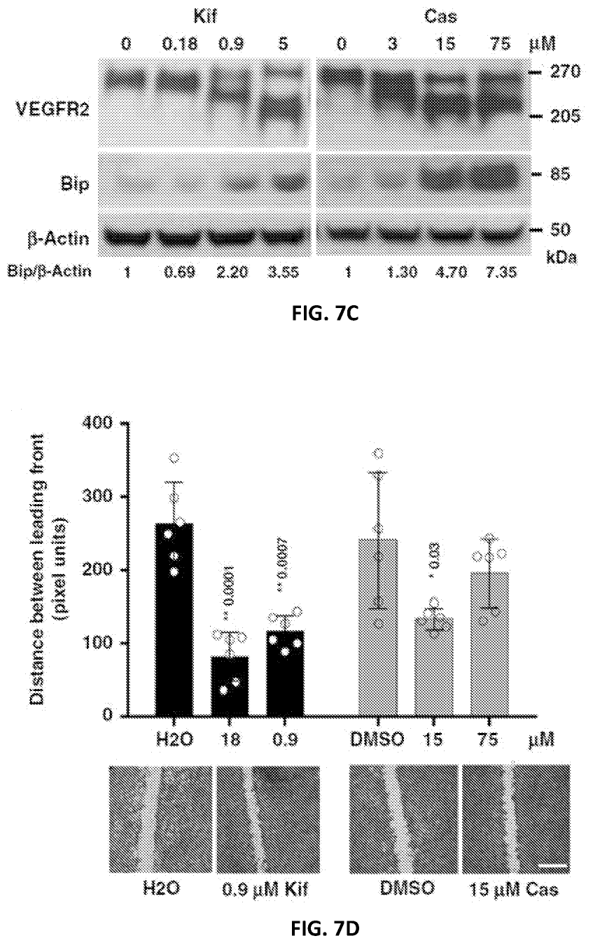

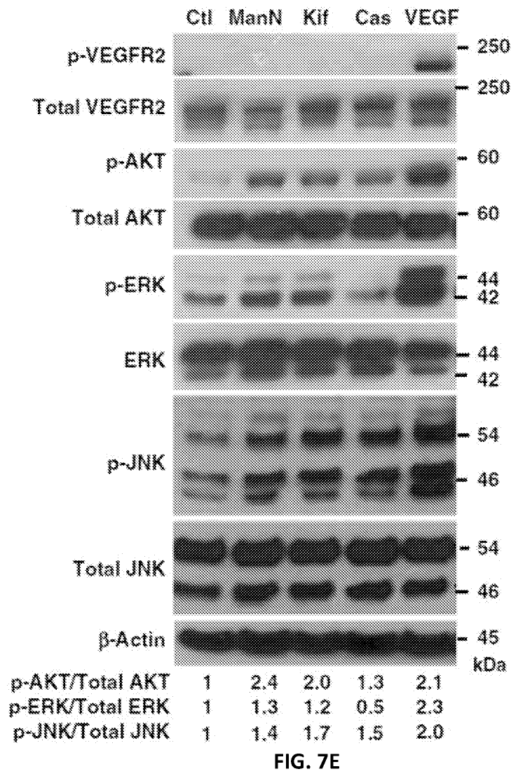

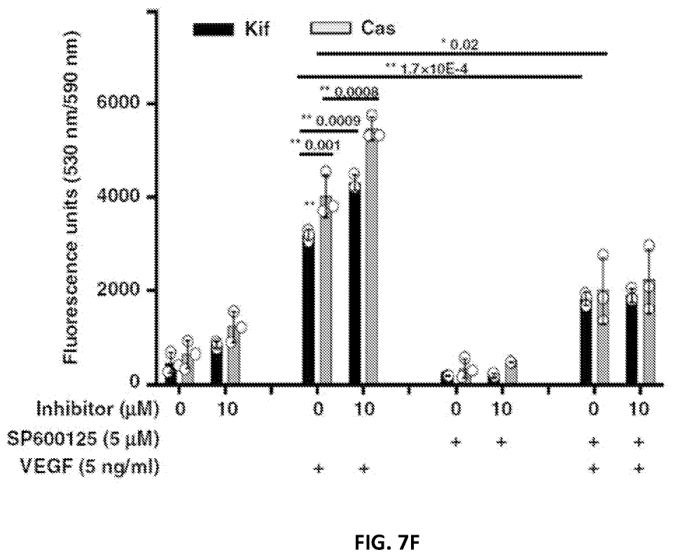

ManN affects protein glycosylation in endothelial cells. The additivity of ManN with VEGF could potentially occur at transcriptional and/or translational level or through signal transduction pathways mediated by VEGF-VEGFR2. However, neither transcription of VEGF, VEGFR2 and GLUT1 & 4 nor total VEGFR2 protein expression was significantly changed in BCECs (FIGS. 4A to 4C; FIGS. 5A and 5B; FIGS. 7C and 7E) when cells were treated with various concentrations of ManN for 4 hours (for gene expression level) or 24 hours (for protein expression level). The same was true for BRECs and hRMVECs. Biotinylation studies showed no changes in the amount of VEGFR2 on cell surface. However, VEGFR2 phosphorylation in response to VEGF was decreased in ManN pre-treated cells, suggesting that VEGFR2 activation was hampered, rather than enhanced in BCECs. No ligand-independent VEGFR2 activation occurred after ManN addition in BCECs. The same was true also for HUVECs (SFIG. 10a) and hDMVECs.

The apparent molecular mass of VEGFR2 shifted significantly following ManN treatment in both BCECs (FIGS. 4A to 4C and 4E) and BRECs, starting at 40 uM. New lower molecular weight bands (˜170-200 KDa) appeared in BCECs treated with ManN in a dose-dependent manner, compared to the control (a major band at ˜230 KDa and a minor band at ˜210 KDa) (FIGS. 4B, C, and E; FIGS. 5A and 5B; and FIG. 7C). This shift was unique to ManN among hexosamines and their derivatives (FIG. 4A). VEGF alone had no effect on molecular mass. Adding VEGF to ManN caused no additional shifts (FIG. 4A). Lower molecular weight VEGFR2 bands are not likely degradation products since ManN removal completely reverse the effects of ManN on molecular mass after 24 hours (FIG. 4E). However, based on PNGase F treatment, it appears that not all the glycosylation on VEGFR2 was abolished by ManN, at least at uM concentrations. Experiments with the small molecule tyrosine kinase inhibitor axitinib, a potent VEGFR2 inhibitor [6], indicate that decreases in VEGFR2 molecular mass and stimulation of BCEC proliferation by ManN are not dependent on VEGFR2 signaling.

A significant change in VEGFR2 protein mass was also observed in hRMVEC, HUVEC (FIG. 4F) and hDMVECs (FIG. 4G) with 40 uM ManN, whereas the additive effect of ManN and VEGF on proliferation of these cells occurred at mM levels.

To better understand how ManN may affect VEGFR2 post translational modification, cells were treated with ManN in the presence of one of four monosaccharides (mannose, glucose, galactose, or fucose) at a maximum 1:10 molar ratio. These monosaccharides are known to be important in protein N-glycosylation. Our results suggest that mannose could dose-dependently block ManN effects on VEGFR2 molecular mass as well as on BCEC proliferation (FIGS. 4C and 4D). The effects of mannose may not be limited to prevention of entry of ManN into cells via the same transporter(s) since the effects were seen when BCECs were first treated with ManN for 2 hours to ensure its successful cellular uptake. Glucose, but not galactose or fucose, had similar effects as mannose.

Decreases in protein mass following ManN administration in BCEC were not limited to VEGFR2. Other N-glycosylated growth factor receptors/co-receptors or adhesion molecules, including αv integrin, Neuropilin-1, VE-cadherin and bFGFR1 were affected as well.

### Example 5

The effect of ManN on general protein glycosylation profile was assessed. N-glycosylation is a complex process, dependent on multiple enzymes that act sequentially on glycoproteins to generate hybrid and high-mannose glycan structures as they transit through the secretory pathway, from ER to Golgi apparatus [22]. It plays an important role in the determination of the fate of newly synthesized glycoproteins in the ER, their correct folding, cellular destination and proper function.

Several key enzymes involved in protein N-glycosylation in both ER and Golgi apparatus were evaluated. α-mannosidase from Jack Bean is a broad-specificity exoglycosidase that catalyzes the hydrolysis of terminal, non-reducing αl-2, αl-3, and αl-6 linked mannose residues from oligosaccharides in both organelles, and controls conversion of high mannose to complex N-glycans, the final hydrolytic step in the N-glycan maturation pathway. This enzyme has been used to screen for potential N-glycosylation inhibitors [23]. ManN, but not other hexosamines or their derivatives, showed inhibitory activity at 400 uM, which is considerably higher than the effective mitogenic concentrations in BCECs. No effect of ManN up to 2 mM on α- or β-glucosidases was detected.

N-linked glycans from BCECs were isolated by enzymatic cleavage, followed by purification and characterization using MALDI-TOF-MS. Treatment with 40 uM ManN resulted in a significant time-dependent reduction in Man6GlcNAc2 (Man-6), Man-8 and Man-9 in total oligomannose N-glycan content compared to the untreated control, whereas a significant early phase accumulation of Man-5 and Man-7 was observed after ManN treatment. ManN has been previously shown to inhibit Lipid-linked oligosaccharide (LLO) synthesis, to change protein GPI biosynthesis and hybrid glycan production as well as to incorporate into the glycans in MDCK cells [24]. Accumulation of Man-5 over time suggested that inhibition of mannosidase is unlikely the mechanism of pro-angiogenic activity in BCECs.

The monosaccharide content was measured to profile the composition of complex N-glycans. A significant decrease in fucose (8 hours), mannose (12 hours), galactose (24 hours) and Neu5Ac (8 hours and 24 hours) were found in ManN-treated cells compared to the untreated control cells, consistent with the inhibitory activity of ManN on overall protein N-glycosylation.

O-glycan modification is another form of post translational modification of proteins, where a serine or threonine residue is covalently linked with a GalNAc residue [22]. The GalNAc residue can be further modified by several glycosyl-transferases acting in a sequential manner to extend the glycan chain, either branched or linearly, according to substrate specificity. The ppGalNAcT (polypeptidyl GalNAc transferase) catalyzes the transfer of a α-GalNAc from UDP-GalNAc to Ser or Thr residue of a glycoprotein, producing the Tn antigen. When the Tn antigen is generated, it can have three different fates: (i) it can be sialylated on C6 by the enzyme ST6GalNAcT; (ii) it can be substituted on C3 or C6 by a β-GlcNAc which gives rise to core-3 or core-6, respectively; or (iii) it can be galactosylated on C3 by the C1GalT1 in order to form core-1 which can also be sialylated to produce mono- or di-sialyl Core-1 O-glycan [22].

O-glycan analysis was conducted in BCEC lysates by MALDI-Tof mass spectrometry. Due to unavailability of a unique enzyme that cleaves all different forms of 0-glycan, a reductive beta-elimination was performed to have an understanding of the O-glycan backbone [25]. To protect from de-sialylation during mass spectral data acquisition, permethylation was performed prior to MALDI-Tof/Tof mass analysis [25]. An overall decrease in O-glycosylation following treatment with 40 uM ManN. In particular, we observed a trend of decrease toward ion intensity at m/z of 895 (Sialyl-Corel, Gal(31-3GalNAc-), 1256 (di-sialylated Core 1), 983 [Core 2, GlcNAcrβ1-6(Galβ1-3)-GalNAc-] and 1187 (di-galactosylated Core 2).

### Example 6

The effect of ManN on activating UPR by increasing Bip and CHOP expression was assessed. Asparagine linked N-glycosylation is one of the most common modification reactions in eukaryotic cells, occurring in proteins that are co-translationally translocated across or integrated into the ER during biosynthesis [22]. After N-linked oligosaccharides are transferred to nascent proteins by the OST (oligosaccharyltransferase), ER resident glucosidases and mannosidases generate a series of glycan-trimming intermediates that are specifically recognized by ER-localized lectins to direct the nascent proteins into protein folding, degradation or export pathways. One of the consequences of inhibition of protein glycosylation is compromised protein folding, leading to ER stress [26, 27]. The physiological responses to the UPR are mediated by changes in gene expression, such as the regulation of ER Hsp70 chaperone BiP (also called glucose-regulated protein 78, binding of immunoglobulin protein) and another multifunctional transcription factor CHOP (CCAAT-enhancer-binding protein homologous protein) [28, 29]. Impaired UPR function, for instance during aging, creates a permissive environment for protein aggregation, unresolved ER stress, and chronic inflammation [30].

To investigate a possible ManN-mediated ER stress, we studied the expression of Bip and CHOP in ManN or mannose-treated cells by western blot analysis. Our data indicate that ManN, but not mannose or VEGF, can significantly turn on Bip expression in a concentration-dependent manner when growing cells are deprived of growth factor supply, with accumulation of Bip being evident at 24 hours (FIGS. 5A and 5B) and 48 hours (FIG. CHOP induction appeared to be faster, at about 6 hours in a dose-dependent manner (FIG. 5A). No synergy between ManN and VEGF in promoting Bip or CHOP expression was noted (FIG. 5B).

We tested two well-known chemical chaperons 4-PBA (4-phenylbutyric acid) [31] and TUDCA (tauroursodeoxycholic acid) to alleviate ER stress in ManN-treated BCECs. Both were previously shown to mitigate Tunicamycin-induced eIF2α-ATF4-CHOP arm of UPR and Bip expression. We found that 2 mM 4-PBA, but not 500 uM TUDCA, could prevent the induction of CHOP expression by ManN at 400 uM and 5 mM and restore the expression of ATF-6 (Activating Transcription Factor-6) by ManN at 400 uM (FIG. 5C). Likewise, restoration of ATF-6 expression was much weaker by TUDCA compared to 4-PBA. As a transmembrane ER glycoprotein, ATF-6 is cleaved liberating a 50 kDa amino-terminal fragment that translocates to the nucleus which activates transcription of ER chaperones and ER-associated degradation components such as Bip and CHOP upon accumulation of improperly folded proteins in the ER, [28]. Pre-treating cells with 1 mM 4-PBA for 4 hours could effectively reverse the bell-shaped activity of ManN on BCEC proliferation in the absence or presence of VEGF. Additivity between ManN and VEGF was largely abolished (FIG. 5D).

### Example 7

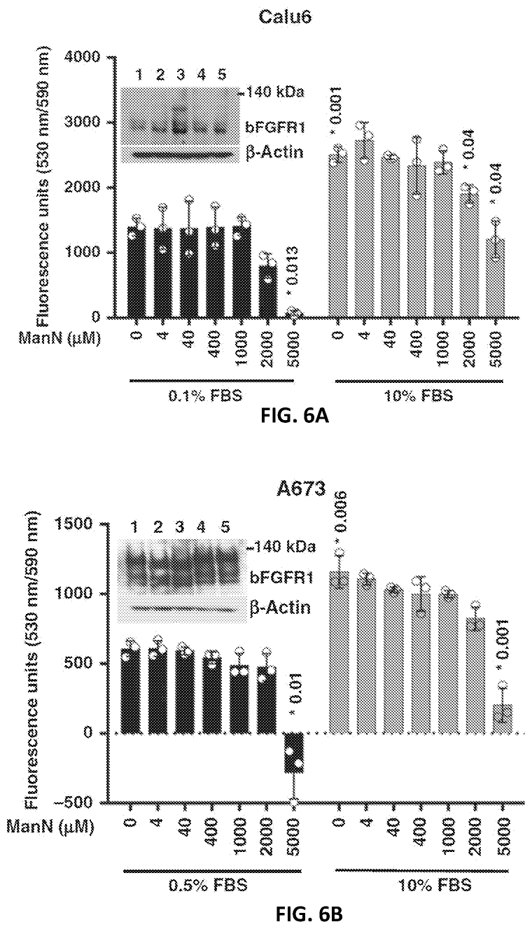

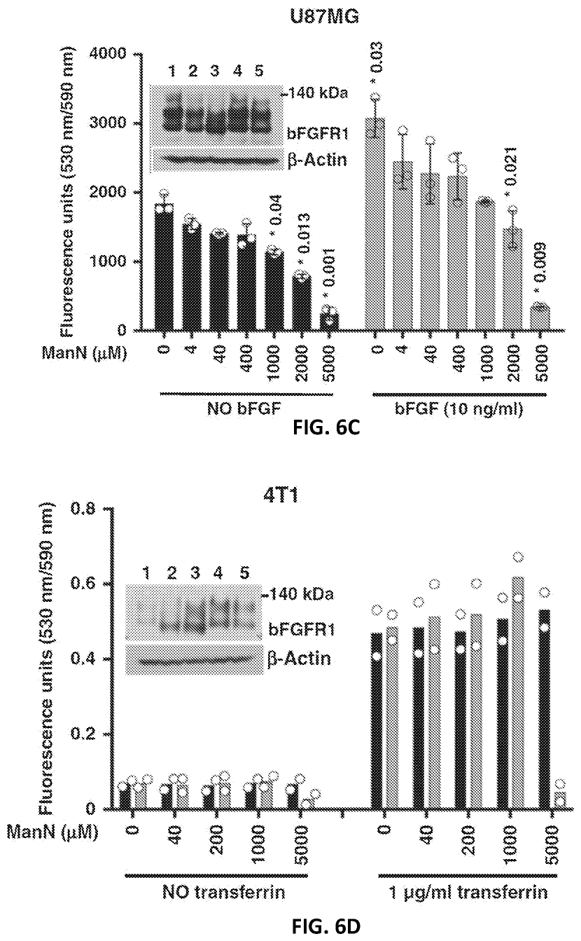

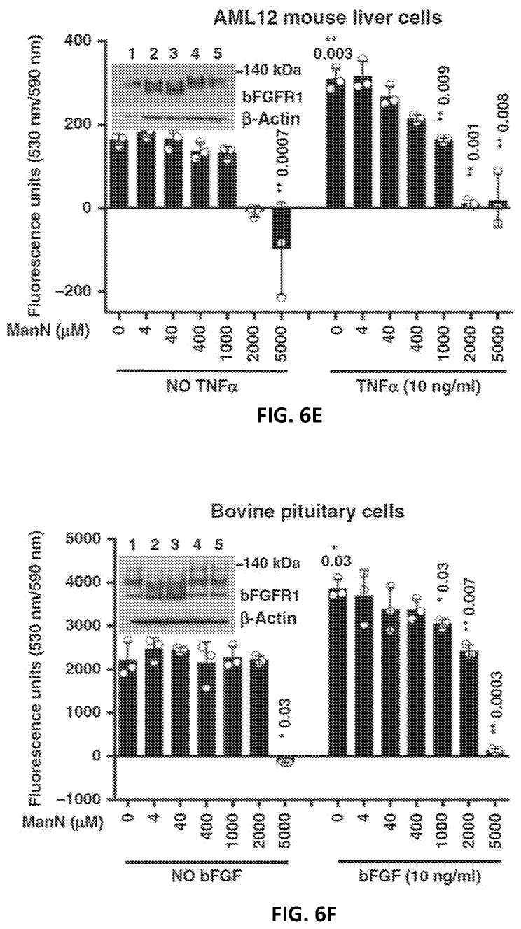

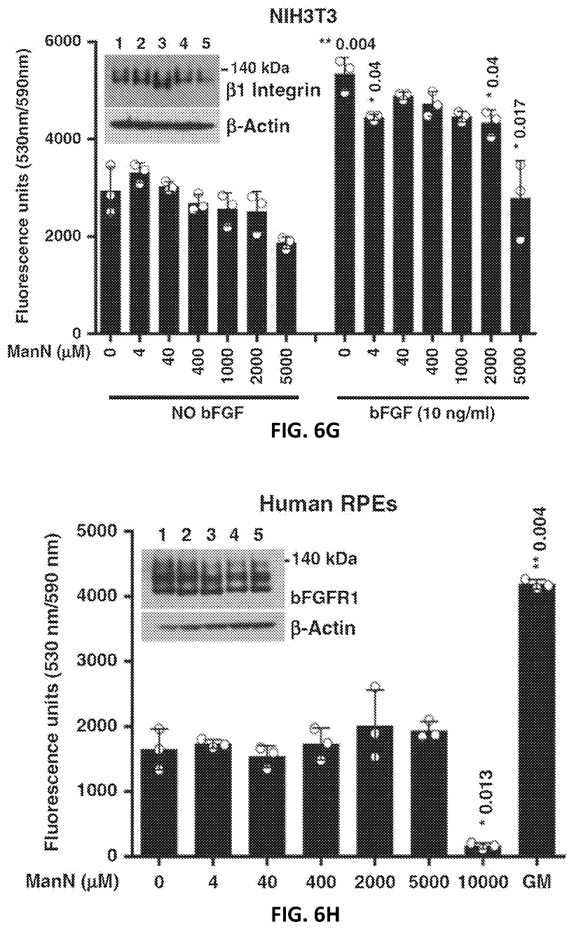

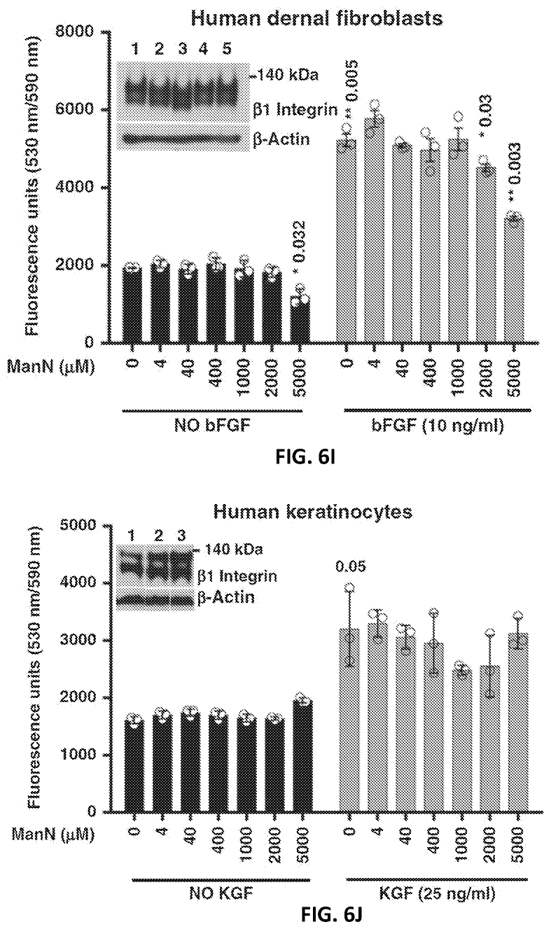

Effects of ManN on non-endothelial cells were assessed. To extend our observations in EC, we examined a variety of non-EC types from different species. These include NIH3T3 fibroblasts and AML12 liver cells (mouse), ARPE-19 RPE cells (human) and freshly isolated bovine pituitary cells. We also tested several human cell types related to our in vivo models such as dermal fibroblasts and keratinocytes. In addition, we screened four human or mouse cancer cell lines (A673, U87MG, Calu6 and 4T1) (FIG. 6). To examine post-translational modifications of proteins in non-ECs, we used bFGFR1 or β1 integrin to monitor molecular mass change. Similar to BCECs, ManN not mannose could induce molecular mass change in all these non-ECs (FIG. 6 inserts). However, unlike BCECs (FIG. 1B), BRECs (FIG. 1C), hRMVECs, HUVECs and hDMVECs, no proliferative effects by ManN were observed at uM to mM concentrations, alone or in combination with other growth stimulators (FIG. 6), although efficient ManN uptake and comparable levels of free ManN were detected in all cell types. Cellular toxicity varied among different cell types, with AML12 being the most sensitive one and human RPE cells and human keratinocytes being the least sensitive ones to 5 mM level of ManN (FIGS. 6E and 6H). Growth inhibition by 25 mM Mannose in vitro has been reported in several tumor lines with low level of PMI (phosphomannose isomerase) [19]. At 5 mM, ManN, but not mannose, showed significant toxicity on 4T1 cells (FIG. 6D), possibly due to a higher PMI level in 4T1 relative to all the reported sensitive tumor lines.

### Example 8

Similar to ManN, inhibitors of protein N-glycosylation stimulate EC growth. To determine whether broad changes in protein glycosylation could promote cell proliferation, two well-characterized inhibitors, Kifunensine (Kif) and Castanospermine (Cas) were tested [33-37]. BCEC proliferation was stimulated in a dose-dependent fashion in the absence or in the presence of 5 ng/ml VEGF (FIGS. 7A and 7B). Following treatment with Kif or Cas for 24 hours, reduction in VEGFR2 molecular mass on SDS-PAGE was evident (FIG. 7C).

At 40 uM, Kif could significantly activate ERK and AKT in BCECs (FIG. 7E), HUVEC and hDMVECs. Activation of ERK by Cas was less obvious in both BCECs and hDMVECs. However, both inhibitors were able to activate the JNK pathway in BCECs (FIG. 7E). Blocking JNK activation with 5 uM SP 600125 significantly reduced the effects of both glycosylation inhibitors on proliferation of BCECs (FIG. 7F). FIG. 7C illustrates a dose-dependent induction of Bip expression when growing BCECs were switched to media without growth factors for 24 hours in the presence of Kif or Cas at concentrations which promoted cell proliferation.

Both Kif and Cas had significant activity in the BCEC “scratch” assay, with gaps being closed more rapidly by each molecules over 48 hours relative to control (FIG. 7D). The inserted panel of FIG. 7D shows representative images from an assay in which Kif or Cas was used. Quantification analysis indicated that there was significant acceleration of gap closing in a dose-dependent manner compared to controls.

### Example 9

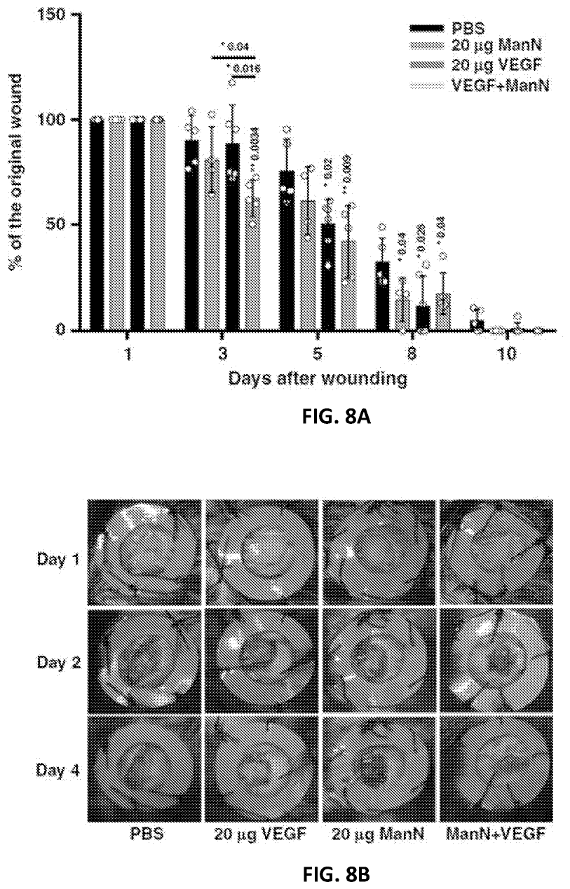

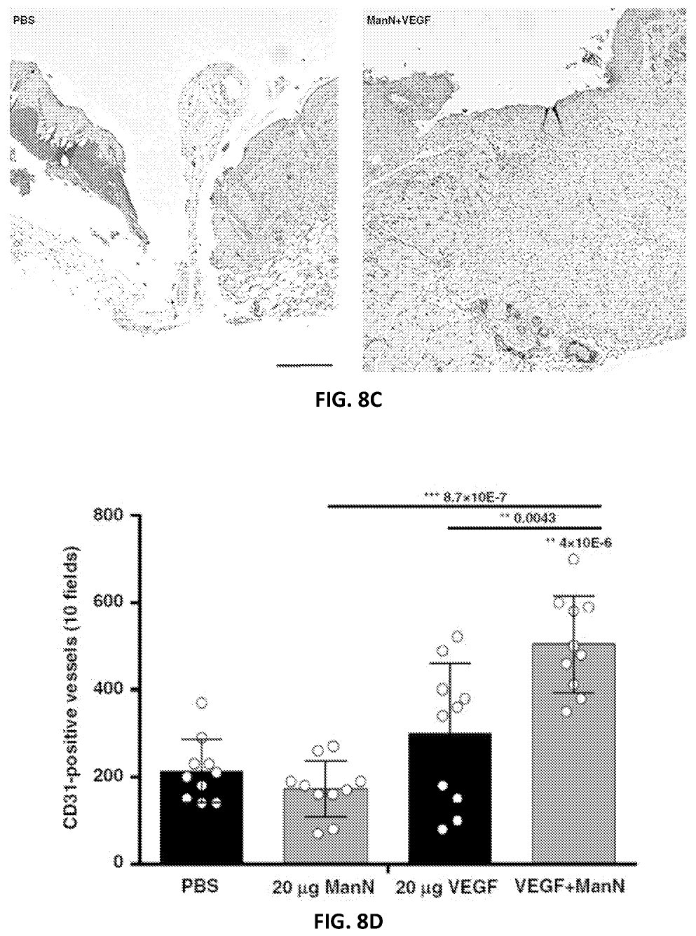

The relation between effects of ManN on endothelial cells in vitro and in angiogenesis in vivo, was investigated via its effects in a splinted wound model in mice. In this model, the repair process is entirely dependent on epithelialization, cellular proliferation and angiogenesis, which closely mirror the biological processes of human wound healing [38]. The effects of ManN and VEGF were tested alone or in combination. Topical applications of 20 μg of VEGF or 20 μg of ManN daily was done for the first 3 days after wounding. When VEGF and ManN were combined, a significant acceleration of wound closure was observed during the early phase of healing (FIG. 8A). Compared with VEGF or ManN monotherapy, the combination had a significant faster wound closure starting from day 2 (FIG. 8B). On day 4, an average closure of the wound was 81.5%, 75.6%, 66.9% and 29.8% in PBS-, ManN-, VEGF- and combination-treated group, respectively. Small vessel numbers were quantified around the wound area at day 4. A significant increase in CD31-positive vessels was found in the combination group compared to PBS control, VEGF or ManN alone (FIGS. 8C and 8D).

Thus, ManN, in combination with VEGF, promotes angiogenesis in a skin injury model. In this acute model, wound closure takes place rapidly, without any treatment.

The stability of ManN was assessed in wound fluid contaminated by bacteria, a common feature of wounds. ManN was added to freshly collected wound fluid from a mouse model of skin infection with Staphylococcus aureus, a prevalent cause of skin and soft tissue infections in humans [39]. No significant loss of free ManN was detected following incubation with such wound fluid for up to 24 hours at 37° C. Thus, ManN may be useful for treatment of infected wounds, possibly in combination with anti-microbials or other agents.

One of the known properties of VEGF is a rapid induction of vascular permeability following injection in the guinea pig skin [1]. The effect of ManN in inducing vascular permeability was assessed in the same assay. However, no permeability-enhancing effects were elicited by ManN when tested at 1 ng-5 μg, while 25 ng VEGF induced vascular permeability.

### Example 10

The angiogenic effects of ManN and Kif in a mouse hindlimb ischemia model were assessed. The activity of ManN in a chronic ischemia model that might more specifically reflect its effects as an endothelial cell mitogen and a pro-angiogenic factor was assessed, and the hindlimb ischemia model in the mouse was considered. Several variants have been described, depending on which vessel is occluded [40, 41]. The variant chosen consists in ligation and excision of the femoral artery, which results in more severe ischemia compared to simple femoral artery ligation [40]. Occlusion of two vessels produces more severe ischemia, but has the disadvantage of inducing severe pain and distress, as well as frequent ulcerations and necrosis in mice [40].

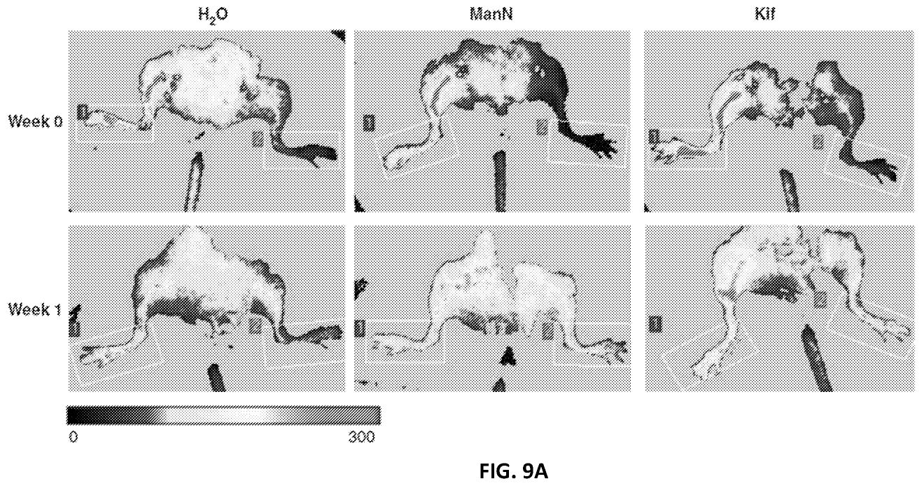

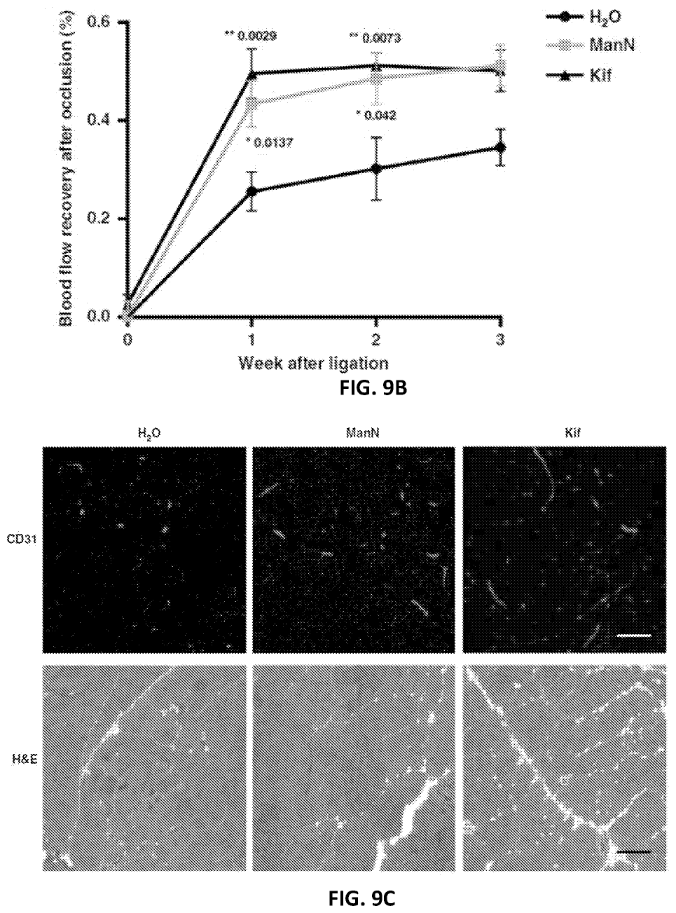

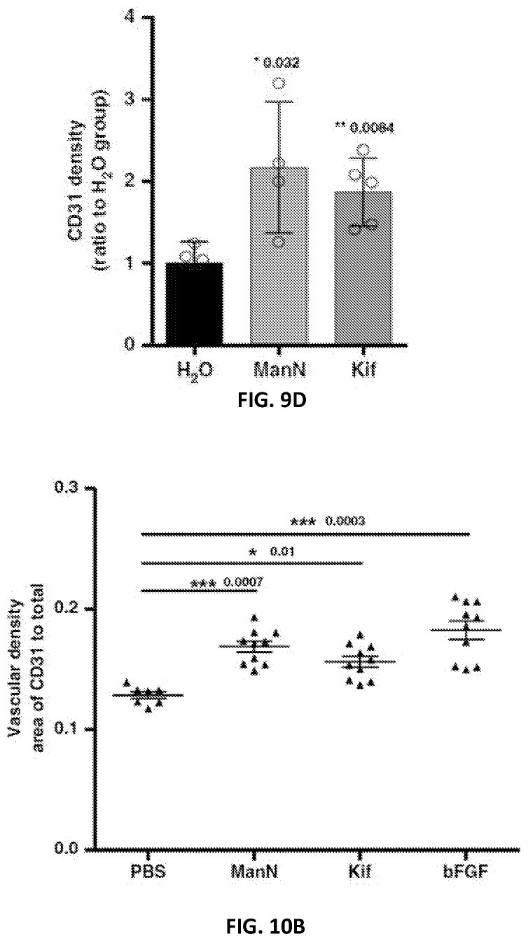

Oral administration of ManN was tested in this femoral artery ligation-excision model. Since Kif has been previously administered intraperitoneally for in vivo studies [42], this route was employed. Laser Doppler Perfusion Imaging (LDPI) was used as a non-invasive method to monitor time and extent of the blood flow recovery in the ischemic limb [43]. Serial examination of blood flow was taken with LDPI and increment of the perfusion ratio of ischemic (ligated; left side) to non-ischemic (sham; right side) hindlimbs after ligation was used to indicate a recovery of blood flow. Starting immediately after surgery, mice were orally fed with 20% ManN or 1 mg/ml Kif ip every other day, as described in methods. One week after surgery, the perfusion ratio in H₂O-fed group indicated a blood flow recovery of ˜25%, a value that is in good agreement with published data with the same type of lesion, in the same strain of mice [40, 44]. However, the blood flow recovery in ManN and Kif-treated group was about 40% and 47%, respectively, which demonstrated an accelerated recovery rate of blood flow compared to H₂O-treated mice (FIGS. 9A and 9B). The blood perfusion ratio continued increasing to ˜50% of sham-treatment limbs in 3 weeks after ManN and Kif treatment and was significantly higher than the control group (FIGS. 9A and 9B).

Consistent with the improved blood flow, the ischemic hindlimbs of ManN-treated and Kif-treated group showed an increased blood vessel density compared to the control group, as assessed by CD31 immunostaining of the surrounding muscle tissue 3 weeks post-ligation. Compared with H₂O-treated control group, blood vessel densities were respectively 2.3 and 1.8 times higher in ManN and Kif-treated groups (FIGS. 9C and 9D).

Following oral administration, there was a relatively rapid decline in ManN plasma levels. Plasma free ManN levels reached a peak level of ˜100 nmol/ml plasma at 1 hr. After 3 hours, only about half of that amount was detectable. 2 hours after oral feeding of 20% ManN, muscle samples were taken from the ischemic legs. A significant amount of ManN reached the ischemic legs, with 0.17+/−0.18 nmol/mg protein of free ManN and 0.91+/−0.24 nmol/mg protein of ManN-6p. At least in BCECs, ManN effects on protein mass lasted for at least 8 hours in the absence of exogenous ManN (FIG. 4E), indicating that even a relatively brief exposure may be adequate to elicit pharmacological effects.

### Example 11

The effect of ManN and Kif on inducing retinal neovascularization was assessed. The findings in cultured eye-derived EC were extended to a suitable in vivo model system. The mouse retina has been used extensively over the past decades to study both physiological and pathological angiogenesis [41]. To obtain a detailed description of the retinal vasculature, images from retinal flat mounts were processed for vascular area fraction (ratio of area covered by blood vessels to total retinal area). Using this model, the effects of ManN in retinal neovascularization were assessed. Kif was also tested in this model because it is a water-soluble inhibitor and its mechanism of glycosylation inhibition is well established [35]. In addition, it shares with ManN the ability to activate ERK, AKT and stress pathways in BCEC (FIG. 7E).

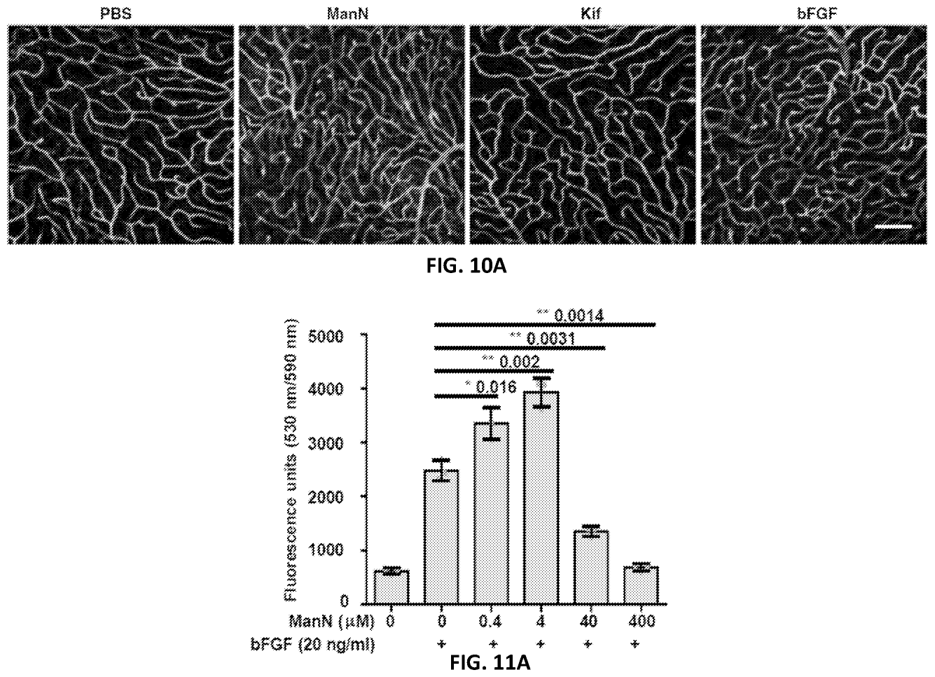

Five hundred nanograms of ManN or Kif was intravitreally injected and the retinal vasculature was examined after seven days. Intravitreally administration of 200 ng bFGF was used as a positive control in this model. In bFGF, ManN and Kif-treated group, the density of retinal vessels was increased by about 35%, 30% and 20%, respectively, compared to PBS group (FIGS. 10A and 10B).

### Materials and Methods

### Small Molecule Library

MSMLS (Mass Spectrometry Metabolite Library of Standards) (IROA TECHNOLOGIES, Bolton, MA; now Sigma) is a collection of 619 high-quality small molecules (purity >95%) that span a broad spectrum of primary metabolites, including carboxylic acids, amino acids, biogenic amines, polyamines, nucleotides, coenzymes, vitamins, lipids, etc. Plates were spun at 300 g after reconstitution, according to the instructions of the manufacturer.

### Chemical Compounds

D-Mannosamine hydrochloride was obtained from Sigma (M4670) or Spectrum Chemical MFG Corp (M3220). 1-Amino-1-deoxy-D-Fructose hydrochloride (D-isoglucosamine) (803278), D-(+)-Galactosamine (1287722), D-(+)-Glucosamine (1294207), N-acetyl-Mannosamine (A8176), N-acetyl-galactosamine (A2795), N-Acetyl-Glucosamine (A8625), Meglumine (M9179), Muramic acid (M2503), N-Acetylneuraminic acid (A2388), D-(+)-Glucose (D9434), D-(+)-Mannose (1375182), Meglumine (M9179), Tunicamycin from Streptomyces sp. (T7765) and SP600125 (S5567) were obtained from Sigma. Hypure cell culture grade water used to dissolve compounds (endotoxin <0.005 EU/ml) was obtained from Hyclone. Axitinib was obtained from Santa Cruz (SC-217679). Tauroursodeoxycholic acid (TUDCA) was from Calbiochem (1180-95-6) and 4-phenylbutyric acid (4-PBA) (P21005), Castanospermine (Cas, C3784), Kifunensine (K1140), DMSO (D2650) were from Sigma. DMSO (D2650) was used as a solvent for Cas.

### Antibodies

Antibodies used in the present study were from Cell signaling Technology Inc. (Danvers, MA) unless otherwise specified. Total: VEGFR2 (2479), ERK (4695), p38 (9212), JNK (9252), mTOR (2983), AKT (4691), CREB (9104), CHOP (2895), ACC (3676), ATF-6 (65880), Bip (3183), AMPK□ (5832), FGFR1 (9740), eNOS (9586), VE-Cadherin (2500), c-Met (3127 or 3148), Neuropilin (3725), CD31 (3528), c-Jun (9165). Phosphor-antibodies: VEGFR2 (Tyr1175, 2478 or 3770), ERK1/2 (Thr202/Tyr204, 4376), p38 (Thr180/Tyr182, 4511), JNK (Thr183/Tyr185, 9251), mTOR (Ser2448, 5536), AKT (Ser473, 4060), CREB (Ser133, 9191), ACC (Ser79, 3661), eNOS (Ser1177, 9571), AMPKα (Thr172, 50081), c-Jun (Ser73, 9164), (31 integrin (4706 & 34971), αv integrin (4711), JNK1 (3708), JNK2 (4672), JNK3 (2305). Anti-β-actin was from Sigma.

### Cells

Primary human umbilical vein endothelial cells (HUVEC, passage 4-10) were obtained from Lonza (C2519AS, Lot #234871) and cultured on 0.1% gelatin-coated plates in endothelial cell growth media (EGM) containing 2% FBS, BBE (Bovine Brain Extract), heparin, human EGF, hydrocortisone, ascorbic acid, GA-1000 (Gentamycin, Amphotericin B) and VEGF. Bovine retinal microvascular endothelial cells (BRECs, #BRMVEC-3) and bovine choroidal microvascular endothelial cells (BCECs, #BCME-4), both from VEC Technologies (Renssellaer, NY), were maintained in fibronectin-coated plates (1 pg/cm 2). The growth medium was low glucose DMEM, supplemented with 10% bovine calf serum (BCS), 5 ng/ml bFGF and 10 ng/ml human VEGF₁₆₅. Cells were maintained at 37° C. in a humidified atmosphere with 5% CO2. bFGF (233-FB) and VEGF₁₆₅ (293-VE) were purchased from R&D systems. Human retinal microvascular endothelial cells (passage <15) was from Cell Systems Corporation (Kirkland, WA). They were grown on 0.1% Gelatin-coated plates in Medium 131 containing 5% fetal bovine serum, hydrocortisone (1 μg/ml), human fibroblast growth factor (3 ng/ml), heparin (10 μg/ml), human epidermal growth factor (1 ng/ml) and dibutyryl cyclic AMP (0.08 mM) (MVGS, S 005-25, Gibco Invitrogen). The human RPE cell line ARPE-19 was from the ATCC. Cells were gently lifted in 0.025% trypsin and plated in RtEGM media (Clonetics) containing 2% FBS, L-glutamine, human bFGF, GA-1000). Once cells attached to plates, serum free RtEGM media was used to maintain the culture for best result. ARPE-19 was obtained from ATCC (CRL-2302) and cultured according to company's instruction. NIH3T3 cells were obtained from ATCC (CRL-1658). Human adult dermal MVECs (CC-2543) were cultured in EGM-2MV (CC-4147, Lonza). Keratinocytes (ATCC, PCS-200-011) were cultured in dermal cell basal media (PCS-200-030) plus keratinocyte growth kit (PCS-200-040). Human primary dermal fibroblasts (ATCC PCS-201-012) were cultured in fibroblast basal medium (ATCC, PCS-201-030) plus growth kit (ATCC, PCS-201-040). Growth stimulators used in the assay included human EGF (R&D systems, 236-EG), murine TGFβ (R&D systems, 410-MT), KGF (Sigma, K1757), or 10% FBS growth media. 4T1 cells were obtained from the ATCC (CRL-2539) and cultured in RPMI-1640 with 10% FBS (Omega Scientific, Tarzana, CA) and antibiotics. A673 (CRL-1598), A549 (CCL-185), U87MG (HTB-14) cells were from ATCC and cultured in high glucose DMEM containing 10% FBS. FBS (S12550) was purchased from R&D systems. BCS (SH30073.03) was obtained from Hyclone. All cell lines used in the study are negative for mycoplasma contamination by various vendors.

### Cell Proliferation Assays

Proliferation assays with BCECs and BRECs were performed [13]. Log-phase growing BCECs or BRECs (passage <10) were trypsinized, re-suspended and seeded in 96-well plates (no coating) in low-glucose DMEM supplemented with 10% bovine calf serum, 2 mM glutamine, and antibiotics (growth medium), at a density of 1200-1500 cells per well in 200 μl volume. All reagents were added at the indicated final concentrations. After 3-6 days, cells were incubated with AlamarBlue for 4 hrs. Fluorescence was measured at 530 nm excitation wavelength and 590 nm emission wavelength. The experiments were repeated at least three times. To create a hypoxic condition, cells were placed in a hypoxia incubator with a mixture of gas consisting of 1% 02, 5% CO2 and 94% Nz. On each 96-well plate, untreated and VEGF-treated (10/ng/ml) wells were included to monitor plate-to-plate variations. 20% methanol or 0.05% DMSO served as negative controls. 0.05% DMSO served as negative controls when Cas was tested in these cells. Human RMVECs and human adult DMVECs were split into Gelatin-coated 96 wells (2000 cells per well) in low glucose DMEM containing 10% FBS. 1200 cells/well was set up for proliferation assay in low glucose media containing FBS. Data was collected at day 4 or 5. HUVEC (p7-10) were grown on gelatin-coated plate until it reached 70-80% confluency.

On the day of the assay, cells were dissociated with 0.05% trypsin, which was neutralized with 0.5% FBS-containing EBM. Cells were briefly spun and then re-suspended in 0.5% FBS-media. Cells were counted and plated in 96-well, 1000 cells/well. Triplicate wells were used for each treatment. Data were collected at day 3, and then cells were fixed in 4% paraformaldehyde for 15 min before adding crystal violet. Cell-covered areas were quantified after taking pictures by Image J software.

Proliferation assays with fibroblasts were done in low-glucose DMEM containing 1% FBS, with or without 10 ng/ml bFGF, or 100 ng/ml human EGF and the assay was ended at day 3. ARPE-19 cells were gently lifted with 0.025% trypsin and plated in RtEGM media (Clonetics) containing 2% FBS, L-glutamine, human bFGF and GA-1000. Once cells were attached to plates, serum-free RtEGM media was used to maintain the cultures. For proliferation assays, 1500 human RPE cells were plated into 96-well plates in low-glucose DMEM containing 1% FBS. A673, U87MG, Calu6 and AML12 cells were grown until confluent and were then harvested and re-suspended in appropriate assay media. For proliferation assays, cells were plated at the density of 1000-2000 cells/well in low-glucose DMEM containing 5% FBS or otherwise stated. Bovine pituitary cells (pituitary folliculostellate cells) were isolated as previously described [67]. For proliferation assays with human epidermal keratinocytes, human DMVECs and human dermal fibroblast cells, 1000 cells/well were plated in low-glucose DMEM containing 1% FBS with or without various growth factors. The assay was ended at day 3 for bovine pituitary cells and at day 4 for all the other cell types. For 4T1, 1000 cells were plated in RPMI-1640 with 2% basement membrane extract (BME) and 2% FBS on BME-coated 96-wells and treated 4 hrs later [68]. Four days later, tumor cell growth was measured by the MTS assay (Promega, Madison, WI), a colorimetric assay that measures metabolic activity of viable cells. Recombinant human transferrin was obtained from EMD Millipore (Temecula, CA). Recombinant mouse apo-transferrin was obtained from Sigma.

### SiRNA Knockdown

BCECs were plated in 6-well culture plates at a density of 1.5×10⁵ cells/well and cultured overnight. 2 ml of antibiotics-free culture medium was used to replace the old medium. siRNAs, including siNegative (Ambion, AM4611), siRNA against JNK1 #2 (Invitrogen, NM_001192974.2_siRNA_266), JNK1 #4 (Invitrogen, NM_001192974.2_siRNA_485), siRNA against JNK2 #2 (Invitrogen, XM_005208371.4_siRNA_1240), JNK2 #4 (Invitrogen, XM_005208371.4_siRNA_696), were mixed with Lipofectamine RNAiMAX reagent (ThermoFisher Scientific, 13778150) in Opti-MEM I Reduced Serum Medium (Gibco, 31985062) according to manufacturer's instructions. Briefly, a mix containing 25 pmol of siRNA, 7.5 μl of RNAiMAX reagent and 125 μl of Opti-MEM medium was used to transfect cells in each well, to a final siRNA concentration of 12.5 nM. A mix of RNAiMAX and Opti-MEM was used as no siRNA control. Cells were incubated with siRNAs. 8 hrs later, the siRNA-containing medium was replaced with fresh medium. 24 and/or 48 hrs after transfection with siRNAs, cells were used for proliferation assays and protein extraction.

### PNGase F Treatment

Glycerol-free PNGase F was obtained from New England Biolabs (Ipswich, MA). Briefly, BCECs were lysed with NP-40 containing proteinase inhibitors (Thermo Scientific, Waltham, MA). Lysates were cleared at 4° C. at 5000 g for 25 mins. Total protein content was measured using Pierce BCA protein assay kit (ThermoScientific). 20 mg of protein was mixed with 10× denaturing buffer and H₂O to a total volume of 10 ml. Glycoproteins were denatured at 100° C. for 10 mins, followed by adding Glycobuffer and PNGase F. The reaction was carried out at 37° C. for 2 hr.

### Western Blots

Cells were allowed to reach ˜80% confluency in 12-well plates. Cells were pre-treated with ManN, Kif or Cas for various time durations, with or without the subsequent addition of VEGF, with H₂O as the solvent control for ManN. At various time points, plates were taken out of the incubator and kept on ice. Cell monolayers were first washed once with ice-cold PBS before lysis with 250 μl of Pierce RIPA buffer (ThermoFisher Scientific, Rockford, IL) or use 50 mM Tris-HCl (pH 7.6), 150 mM NaCl, 10% Glycerol, 1% NP-40 containing protease/Phosphatase inhibitor cocktail (100×) (Cell signaling, #5872). Lysates were collected and mixed with 4×Bolt LDS Sample Buffer (Novex, Carlsbad, CA) in the presence of Halt protease inhibitors and phosphatase inhibitor cocktail (ThermoFisher scientific, #NP0007). The samples were subjected to SDS-PAGE (Bolt 4-12% Bis-Tris Plus, Invitrogen) using Bolt MES SDS running buffer or NuPAGE 3-8% Tris-Acetate gel using Tris-Acetate SDS running buffer (Novex). HUVECs (passage 6-8) were plated in EBM-2 basal medium (Lonza) with 0.2% FBS. Following overnight culture, cells were serum-starved in EBM-2 medium for 4 hrs prior to treatment with 50 ng/ml of VEGF 165 or vehicle controls for various lengths. Equal amounts of protein lysates were analyzed by SDS-PAGE and blotted with the indicated antibodies. Proteins were transferred using Tris-Glycine buffer with 20% Methanol (Proteonomics grade) (Apex BioResearch Products). Membranes were first incubated with 5% milk in TBST, pH 7.6 (TEKnova, Hollister, CA), followed by blotting with primary and secondary antibodies. ECL anti-rabbit IgG, horseradish peroxidase linked whole antibody from donkey or sheep anti-mouse were obtained from GE Healthcare (UK limited). SuperSignal West Dura Extended Duration substrate was from ThermoFisher Scientific. In some cases, the same PVDF membranes were stripped by 8-min incubation in the Restore Plus Western Blot Stripping Buffer (ThermoFisher Scientific) to show total specific protein expression, followed by second stripping for β-actin expression.

### Migration Assays

HUVECs (passage 6-8) were cultured and serum-starved as described in “Western Blots”. Ten thousand cells in 150 μl of EBM-2 medium were then added to the upper chamber of 8 μm pore size cell culture inserts (Falcon) coated with 0.1% gelatin. The lower compartment was filled with 600 μl EBM-2 medium containing various agents. The plates were incubated at 37° C. to allow migration. After 4 hrs, cells were fixed with 4% PFA for 20 min and then stained with crystal violet (Sigma-Aldrich) for 20 min at RT. Migrated cells on the bottom side of the insert membrane were quantified by counting whole area of the insert at 40× magnification. The experiments were carried out in triplicate and repeated three times. BCEC migration was set up similarly, except that wells were coated with FN, cells were suspended in 1% serum media and migration time was 18-24 hrs.

### Scratch Assay

BCECs (passage 6-10) and HUVEC (passage 6-8) were used in this assay. Cells were grown until about 80% confluency in 6-well plates, washed twice with PBS and then starved in serum-free DMEM (low Glucose, Hyclone) for 5 hrs before making a “scratch” using 1 ml tip. Cell monolayers were briefly washed once with serum-free media, followed by various treatments in media containing 1% FBS. 48 hrs later, the assay was stopped by adding 2 ml 4% paraformaldehyde. 20 min later, fixed cells were stained with 1 ml Crystal Violet (Sigma). Plates were washed gently under the running tap water and air-dried before taking pictures. Images were acquired by ZEISS Discovery V8 SteREO microscopy equipped with PixeLINK Megapixel FireWire camera. Quantification of wound closure was done using AxioVision LE Rel.4.4 software. Six images were taken for each sample and six measurements (in pixel) were made on each image using AxioVision LE Rel.4.4 software.

### N-Glycan, Monosaccharide, Sialic Acid, 0-Glycan Analysis

As soon as BCECs reached about 80% confluency, they were washed twice with phosphate buffered saline (PBS, Sigma) and harvested by scraping. The cells were pelleted by centrifugation at 300 g for 3 mins and washed once with cold PBS. Cells were homogenized and total protein was measured. All subsequent analysis was based on known protein amount.

N-linked glycans were removed from glycoprotein samples using PNGase-F kit (New England BioLabs, P0705S). Briefly, 300 μg of protein sample was reconstituted in 180 μl UltraPure water. 20 μl of 10× denaturing buffer was added and boiled using 100° C. water bath for 14 mins. Samples were cooled down to room temperature and centrifuged at 2700 g for 1 min. Subsequently, 50 μl 10×NP-40 was added and samples were kept at room temperature for 30 mins with vortexing at 5 min interval, followed by adding 25 μL of 10×reaction buffer and mixing thoroughly. 5 μl PNGaseF (2500 U) was then added to the samples and mixed gently. Samples were incubated at 37° C. for 16 hrs. Released N-glycans were purified using solid phase extraction method. Briefly, N-glycans were purified by passing the reaction mixture sequentially over pre-conditioned Sep-Pak C18 1 cc cartridge (Waters) and HyperSep PGC (poly graphitized charcoal) cartridge (25 mg, 1 ml Thermo Scientific). The cartridge was washed with 4 ml of water and the PGC alone was washed with additional 1 ml of water. N-glycans bound to PGC were eluted using 30% acetonitrile containing 0.1% TFA in water. Finally, purified N-glycans were lyophilized and labeled with 2-AB. Briefly, samples were dissolved in 10 μl solution of 0.44 M 2-AB (2-Amino benzamide) in 35% acetic acid in DMSO containing 1M sodium cyanoborohydride. The samples were incubated at 65° C. for 2.5 hrs. The 2-AB labeled glycans were purified using GlycoClean S cartridge (GLYKO) following their glycan clean-up protocol. Excess reagent was removed from the samples using Glycoclean S-cartridge (Prozyme) and labelled glycans were dried using SpeedVac and stored at −20° C. Profiling of 2-AB labeled glycans was obtained using Dionex CarboPac PA1 (4×250 mm) anion exchange column along with a guard column (4×50 mm) at flow rate of 1 ml/min. Glycans were separated in 100 mM sodium hydroxide with a sodium acetate gradient of 0-250 mM in 0-75 minutes. The data was collected using the Dionex ICS-3000 HPLC system with Ultimate 3000 fluorescence detector (Dionex) set at λₑₓ 330 nm at tem 420 nm with sensitivity 7. The data was processed using Chromeleon software (Thermo Scientific).

Monosaccharide composition analysis was done using HPAEC-PAD (Thermo-Dionex ICS3000) and nmole amount of each monosaccharide present in 25 μg of protein was calculated. Samples were hydrolyzed using 2 N trifluoroacetic acid (TFA) at 100° C. for 4 hrs. Followed by removal of acid using dry nitrogen flush. To ensure complete removal of acid, samples were co-evaporated twice with 100 μl of 50% isopropyl alcohol (IPA). Finally, the samples were dissolved in Milli-Q water and injected on HPAEC-PAD. Monosaccharide profile was done using Dionex CarboPac™ PA1 column (250 mm×4 mm; with 50 mm×4 mm guard column). An isocratic solvent mixture of 19 mM sodium hydroxide with 0.95 mM sodium acetate was used at a flow rate of 1 ml per minute for 25 mins. Data were acquired using manufacture supplied standard Quad waveform for carbohydrates. All neutral and amino sugars were identified and quantified by comparing with authentic monosaccharide standard mixture consisting of L-fucose, D-galactosamine, D-glucosamine, D-galactose, D-glucose and D-mannose [70].

Mild acid hydrolysis was used to release sialic acid. Briefly, samples were treated with 2 M acetic acid at 80° C. for 3 hrs followed by removal of excess acid using speed vacuum. Sialic acid was then tagged with DMB reagent and analysis was done using RP-UPLC-FL (Waters Acquity UPLC) system. Known amount of standard Neu5Ac was used to quantify amount of sialic acid in samples.

For O-glycan analysis, homogenized cell samples were treated with 50 mM NaOH in presence of 1M NaBH 4 for 16 hrs at 45° C. The reaction mixture was neutralized using ice-cold 30% acetic acid slowly. The neutratized reaction mixture was then passed over Dowex cation exchange resin to remove sodium ion and lyophilized. Excess boric acid generated during neutralization was then removed by co-evaporation using acidified methanol and methanol respectively. Finally the O-glycan was purified by passing over C18 cartridge. Dried and purified O-glycan was then methylated and used for O-glycan analysis after permethylation. Permethylated samples were then dissolved in absolute methanol and mixed with SDHB (Super-DHB) MALDI matrix in 1:1 v/v ratio and spotted on maldi plate. Mass spectral data was acquired using Bruker AutoFlex mass spectrometer at positive, reflectron mode. The mass spectral data were analyzed and annotated using GlycoWork Bench software and masses matched with the proposed structures were annotated. The mono-isotopic ion intensities are taken for calculation.

To measure cellular uptake of ManN and subsequent conversion to ManN-6P, BCECs were grown in 60 mm dishes to a density of ˜6×10⁵ cells per dish. ManN was added to cultures at the final concentration of 400 μM. Cells were then incubated for 2 hrs. Monolayers were washed three times with PBS at room temperature and lifted by a cell scraper on ice in 10 ml PBS. Cell pellets were obtained by centrifugation at 400 g for 5 mins and stored at −80° C. for further use. Cell pellets were suspended in 200 μl of Ultra-pure ice-cold water in presence of 1 μl of protease inhibitor. Cells were sonicated for 1 min with 30 sec pulses and vortexed to form homogeneous solution. 2.5 μl of the homogenate was used for protein estimation using BCA-assay method in triplicate. A standard curve of BSA at concentration between 0-800 μg/ml was done to quantitate the total protein amount. The cell homogenate was filtered through pre-washed 3K filters and the filtrate was dried using speed-vac. The dry sample was reconstituted in 100 μl of ultrapure water and sample with 200 μg equivalent of protein was injected onto HPAEC-PAD. A known amount (1 nmol) of ManN, Glucose, Mannose, and ManN-6P standards were used to quantify the sugars present in the samples. All standards, except ManNH₂-6P, were obtained from Sigma-Aldrich. ManNH₂-6P was from Omicron Biochemicals, Inc (South Bend, IN). The amounts of monosaccharides present in different cells are presented as nmol/mg of total protein amount. All analyses was performed in a Thermo-Dionex ICS system using a CarboPac-PA-1 column in 100 mM NaOH and 250 mM NaOAc as HPLC running buffer.

### Biotinylation of Surface Proteins

BCECs were plated in 10 cm cell culture dishes 3 days prior to the cell surface protein isolation. Cells were washed three times with Dulbecco's PBS with CaCl₂ and MgCl₂, followed by a 30 min incubation with EZ-Link Sulfo-NHS-SS-Biotin (Pierce, Rockford, IL, USA; 0.5 mg/ml in Dulbecco) on ice. Cells were washed twice with Dulbecco and the non-reacted biotin was blocked with 20 mM glycine for 15 mins. To prevent the reduction of the disulfide bridge in the biotin molecule during the cell lysis process, a 100 uM oxidized glutathione (Sigma-Aldrich, St. Louis, MO) was added in the last wash solution. For cell lysis 500 □1 of lysis buffer (2% NP-40, 1% Triton X-100, 10% glycerol, 100 uM oxidized glutathione, EDTA free protease inhibitor tablet (Roche, Mannheim, Germany) in PBS was added to the cells. Lysed cell extracts were scraped off the plates and transferred to an Eppendorf tube followed by incubation on ice on a shaker for 30 mins. The cell extracts were incubated with 30 U of DNase (22° C. 50 mins, Roche, Mannheim, Germany) and centrifuged for 20 mins (20,800×g, at 4° C.) to pellet the insoluble material. The protein concentration of the supernatants was determined. Equal amounts of protein (˜2 mg) from each extract were used for cell surface protein isolation. The supernatant was pre-cleared using biotin agarose beads (Pierce ImmunoPure Immobilized D-biotin, Thermo Scientific, 20221) and pre-cleared solution was used for the cell surface protein isolation using streptavidin beads. Beads were washed four times with the lysis buffer, four times with 300 mM NaCl in lysis buffer and twice with 50 mM Tris—HCl, pH 7.8. Proteins were eluted twice with an elution buffer (50 mM DTT in 50 mM Tris—HCl, pH 7.8) at 30° C., followed by pooling of the elutes. Three biological replicates and one non-biotinylated control were used in the study.

### Gene Expression Analysis by Real-Time Q PCR

RNA was prepared using the RNeasy Mini Kit (Qiagen). Fifty ng of total RNA per reaction was used for the real-time PCR (Taqman) analysis. Reactions were set up in MicroAmp Fast Optical 96-well reaction plate, seal with MicroAmp optical Adhesion film and run on ViiA7 Real time PCR system (Applied Biosystems) and the absolute quantification with standard curve was used with Sequence Detection System (SDS) software. The expression level of each gene was further quantified relative to the housekeeping gene RPL19 in the same sample. Taqman primers and probe mixes were obtained from Thermo Fisher Scientific. Bovine VEGF-A (Bt03213282), bovine RPL19 (Bt03229687) and bovine specific VEGFR2 (Bt03258877), GLUT1 (Bt03215313) and GLUT4 (Bt03215316).

### α-Mannosidase, α- and β-Glucosidase Activity Assays

a-mannosidase activity was measured using substrate p-nitrophenyl α-mannopyranoside (1 mM). Enzyme from Jack Bean (M7257) (final concentration of 0.077 U) was incubated at 37° C. in a final volume of 50 μl of 50 mM potassium phosphate buffer, pH 7.5. α-glucosidase was assayed with substrate p-nitrophenyl α-glucoside (7 mM). Enzyme from Saccharomyces cerevisiae-type 1 (Sigma, G5003) (final concentration of 0.1 U) was incubated at 37° C. in a final volume of 50 μl of PBS, pH 7.5. β-Glucosidase was assayed with substrate 4-nitrophenyl β-D-glucopyranoside (Roche). Enzyme from almond (Sigma, G0395) (final concentration of 0.002 U) was incubated at 37° C. in a final volume of 50 μl of PBS, pH 7.5 containing 1% SDS. The incubation was stopped by addition of an equal volume of acid-based stop solution (R&D systems, 895032). Enzymatic activity was measured at 405 nm. α-glucosidase from Saccharomyces cerevisiae type I (G5003) with p-nitrophenyl α-D-glucopyranoside (Sigma, N1377) as the substrate.

Measurement of ManN in Wound Fluid from S. aureus Infected Mice

To test ManN stability in wound fluid collected from mice with skin infection as described below, 1.5 μl of 5% ManN solution was added to each 200 μl wound fluid which was first diluted 1:1 (v/v) with PBS. At each time point, samples were taken from 37° C. incubator and stored at −80° C. Plasma proteins were precipitated by adding ice cold acetonitrile to plasma: acetonitrile 1:3 (v/v) ratio. Samples were kept over ice for 1 hr and then centrifuged at 12000 g for 10 min at 7° C. to form a pellet. Supernatants were transferred to other tubed, dried down on a Speed Vac and then reconstituted in UltraPure distilled water and filtered through pre-washed Nanosep 3K Omega filters (Pall Corporation). The filtrate was dried down on Speed Vac. The dry samples were dissolved in 100 μl of water and 2 μL plasma or wound fluid sample was subjected to HPLC analysis. Neutral and amino sugars were separated on a Dionex CarboPac™ PA1 column 4 mm×250 mm with 4 mm×50 mm guard column. An isocratic gradient of 19 mM sodium hydroxide with 0.95 mM sodium acetate was used at a flow rate of 1 ml/min with 20 min run. Data was collected using the Dionex ICS-3000 HPLC system with pulsed amperometric detector using standard Quad waveform. ManN was identified and quantified by comparison with monosaccharide standard using Thermo Scientific Chromeleon software. No ManN samples served as negative controls.

### Skin Wound Healing Model

All animal experimental procedures were approved by the Institutional Animal Care and Use Committee (IACUC) of the University of California San Diego and conducted in an ethical fashion and in accordance with the guidelines of the Animal Care Program (ACP).

The model has been previously described [38]. Briefly, C57BL/6 female mice (8-10 weeks old) were obtained from Jackson labs (Sacramento, CA). A fresh, full-thickness punch wound (4 mm diameter) using a punch (Acu Punch, Acuderm inc. Ft. Lauderdale, FL) splinted with a sterile neoprene ring (6-mm outer diameter and 4-mm inner diameter), fastened with 5-6 sutures (4-0 nylon) under the influence of Isoflurane was created on the back of the animal in a Class II Biological Safety Cabinet. For all surgical procedures, sterile technique was followed. Buprenorphine was given subcutaneously prior to awakening from anesthesia for anticipated pain. Mice were monitored until fully awake and were housed individually to minimize damage/biting/fighting to the surgical site. Recombinant human VEGF was a gift from Roche-Genentech (Telbermin, recombinant human VEGF₁₆₅). Treatment agents were prepared in PBS, sterile-filtered and 25 μl solution was applied daily directly to a wound bed for the first 4-5 days under the influence of Isoflurane, followed by daily observation. Wound closure was monitored by regular imaging and the wound area was quantified using ImageJ (National Institutes of Health, Bethesda, MD, USA).

At day 4 after wounding, wounds were excised with a 2 mm rim of surrounding tissue and placed in 10% formalin for a maximum of 24 hrs. The wounds were then bisected down the center, and 5-1.1m paraffin sections were processed for Hematoxylin and Eosin (H&E) and Masson's Trichrome staining. Epithelial gap was measured histomorphometrically using AxioVision LE Rel.4.4 software. The skin tissues were fixed in 10% formalin for 24 hrs. Paraffin embedded and sectioning were performed by the UCSD, Moores Cancer Center Histology Core. The 5-1.1m paraffin sections were deparaffinized and rehydrated before heat-induced antigen retrieval was performed in 10 mM citrate buffer (pH 6.0). Immunostaining was performed as previously described [20]. Anti-CD31 (SZ31, rat IgG2a) (Dianova, Warburgstrasse 45, 20354 Hamburg, Germany) was used at 2 μg/ml. Small vessels stained positive for CD31 were counted microscopically on 10 fields (20×) taken around the wound.

### Vascular Permeability Assay

Vascular permeability was assessed using a modified Miles assay [14]. Hairless male guinea pigs (Crl: HA-Hrhr/IAF, 75 days old, 450-500 g, Charles River Laboratories) were anesthetized by intraperitoneal (i.p.) administration of xylazine (5 mg/kg) and ketamine (75 mg/kg). The animals then received an intravenous injection (penile vein) of 1 ml of 1% Evans blue dye. After 15 mins, intradermal injections (0.05 ml/per site) of different doses of ManN were administrated into the area of trunk posterior to the shoulder. All reagents were diluted in PBS for intradermal administration. 25 ng of VEGF₁₆₅ per site was used as positive control. 30 mins after the intradermal injections, animals were euthanized by i.p. injection of pentobarbital (200 mg/kg). Skin tissues were dissected from the connective tissues and photographed.

### Murine Skin Infection Model

A skin infection model in the mouse was established [39]. Briefly, mid-log phase of Staphylococcus aureus sub-cultured from overnight cultures in Todd Hewitt broth were used in this study. 6-8 weeks old C57BL/6 mice were obtained from Charles River Laboratories. Mice were shaved and depilated by Nair cream before infection. 5×10⁷ CFU of S. aureus was intradermally injected into the left groins of mice. After 3 days, abscesses were surgically removed and homogenized on ice. Fluid was collected and spun at 14000 rpm. Cleared supernatant was diluted with PBS 1:1 for further use. Animals were housed in clean cages and experimental procedures hereafter were carried out under pathogen-free conditions. The presence of bacteria in the wound fluid was confirmed using Todd Hewitt Broth (THB) plates.

### Hindlimb Ischemia Model and Evaluation of Blood Flow

C57BL/6 male mice (6-8 weeks old) were subjected to unilateral hindlimb surgery under anesthesia with ketamine/xylazine cocktail [41, 43]. Briefly, the left femoral artery was separated from the vein and nerve, ligated proximally, and excised. The right hindlimb served as control. Blood flow was measured by using a laser Doppler perfusion imager (PeriScan PSI; Perimed). Ischemic and nonischemic limb perfusion was measured before and after surgery and 1, 2 and 3 weeks later. After surgery, mice were randomly allocated to different groups (8 mice for each group). 200 μl of 20% ManN was orally administrated every other day from 3^{rd} day after surgery. 200 μl of 1 mg/ml Kif was administrated through ip injection every other day. H₂O was used as a vehicle control. The final blood flow values were expressed as the ratio of ischemic to nonischemic hindlimb perfusion from the same animal. Quantification of blood vessel area was carried out as described [41, 43].

### Retinal Neovascularization

Assessment of retinal angiogenesis following intravitreal administration was done [41]. Briefly, 6-8 weeks old C57BL/6 male mice were randomly allocated to different groups and anesthetized with ketamine/Xylazine cocktail. The indicated amounts of ManN, Kif or bFGF (R&D systems, AF-233-NA) in 1 μl of PBS and PBS vehicle control were injected intravitreally with a 33-gauge Hamilton syringe. Seven days after injection, animals were euthanized. Eyes were then enucleated and fixed in 4% paraformaldehyde (PFA) for 30 mins. Retinas were separated and stained with anti-CD31 immunofluorescence (IF) to evidence the vasculature. Evaluations were performed by an investigator blinded to the treatment. For CD31 IF, rat anti-mouse antibody (BD Biosciences, CAT #550274) was diluted 1:100 and incubated overnight at 4° C. After 4-hour incubation with the Alexa Fluor-488-conjugated anti-rat antibody (Life Technologies, A11006), whole mounts were imaged via the 488 nm channel using A1R Confocal STORM super-resolution system (Nikon). Quantification of vascular density in choroids and retina was carried out by Image J. Each experiment was repeated three times with similar results, and each treatment group consists of 5 individual samples.

### Statistics and Reproductivity

Statistical parameters including the n values, are indicated in the figure legends. The sample size was determined to ensure adequate power, as recommended by the Biostatistics and Bioinformatics Department, Moores Cancer Center. We used 2-tailed, two-sample unequal variance t test. Statistical significance was further confirmed using Wilcoxon rank-sum test between treatment groups of interest for some of the in vitro data sets as the method does not need the normal assumption on variables. Statistical inference was based on the p-value of each comparison using R function “wilcox.test”. We used linear mixed effects (LME) model to investigate the wound area (in percentage), between three treatment groups (ManN, VEGF, VEGF+ManN) and PBS group. Two LME models were fitted. In the first LME model, the controlled group was considered as the reference group. We included day effect (considered days as categorical variable instead of continuous variable), and its interaction with treatment as fixed effects, and we included subject id as the random effect to involve correlations among measurements on different days for the same subject. There's no difference among different groups at baseline. In the second LME model, we relevel group VEGF+ManN as the reference group in order to investigate comparisons between single treatment groups and combination treatment. For each LME model, we explored different treatment effects and their corresponding p-values on Day 3, Day 5 and Day 8 with respect to the reference group respectively. Data are considered significant when p<0.05. Significant p values are represented in the figures as follows: ***p<0.001, **p<0.01, *p<0.05. For each experiment, a representative experimental result is shown from 2-5 independent studies.

### Aspects

Aspect 1: A method of treating an ischemic condition in a subject, the method including administering to the subject in need thereof an effective amount of hexosamine D-mannosamine (ManN).

Aspect 2. The method of aspect 1, wherein the administration is effective to promote endothelial cell proliferation and angiogenesis in the subject.

Aspect 3. The method of aspects 1 or 2, wherein the method further includes administering to the subject in need thereof an effective amount of an N-glycosylation inhibitor.

Aspect 4. The method of any of aspects 1 to 4, wherein the method further includes administering to the subject in need thereof an effective amount of VEGF.

Aspect 5. The method of any of aspects 1 to 5, wherein the ischemic condition is caused by a disease or a trauma.

Aspect 6. The method of any of aspects 1 to 6, wherein the administration is intravenous, intraperitoneal, or intravitreal.

Aspect 7. A method of inducing angiogensis in a subject, the method including administering to the subject in need thereof an effective amount of hexosamine D-mannosamine (ManN).

Aspect 8. The method of aspect 7, wherein the administration is effective to reduce ischemia in the subject.

Aspect 9. The method of aspects 7 or 8, wherein the method further includes administering to the subject in need thereof an effective amount of an N-glycosylation inhibitor.

Aspect 10. The method of any of aspects 7 to 9, wherein the method further includes administering to the subject in need thereof an effective amount of VEGF.

Aspect 11. The method of any of aspects 7 to 10, wherein the subject is in need of inducing angiogenesis is due to an ischemic condition caused by a disease or a trauma.

Aspect 12. The method of any of aspects 7 to 11, wherein the administration is intravenous, intraperitoneal, or intravitreal.

Aspect 13. A method of inhibiting protein glycosylation in a cell, the method including administering to the cell an effective amount of hexosamine D-mannosamine (ManN).

Aspect 14. The method of aspect 13, wherein the administration is in vivo.

Aspect 15. The method of aspects 13 or 14, wherein the administration is ex vivo.

Aspect 16. The method of any of aspects 13 to 15, wherein the administration is effective to stimulate EC proliferation and angiogenesis.

Aspect 17. The method of any of aspects 13 to 16, wherein the administration is effective to activate JNK and an unfolded protein response caused by ER stress.

Aspect 18. The method of any of aspects 13 to 17, wherein the administration is effective to induce changes in N-glycan and O-glycan profiles.

### REFERENCES

1. Apte, R. S., D. S. Chen, and N. Ferrara, VEGF in Signaling and Disease: Beyond Discovery and Development. Cell, 2019. 176(6): p. 1248-1264.
2. Chung, A. S. and N. Ferrara, Developmental and pathological angiogenesis. Annu Rev Cell Dev Biol, 2011. 27: p. 563-84.
3. Potente, M., H. Gerhardt, and P. Carmeliet, Basic and therapeutic aspects of angiogenesis. Cell, 2011. 146(6): p. 873-87.
4. Ferrara, N. and K. Alitalo, Clinical applications of angiogenic growth factors and their inhibitors. Nature Medicine, 1999. 5(12): p. 1359-1364.
5. Simons, M., Angiogenesis: where do we stand now? Circulation, 2005. 111(12): p. 1556-66.
6. Ferrara, N. and A. P. Adamis, Ten years of anti-vascular endothelial growth factor therapy. Nat Rev Drug Discov, 2016. 15(6): p. 385-403.
7. Munkley, J. and D. J. Elliott, Hallmarks of glycosylation in cancer. Oncotarget, 2016. 7(23): p. 35478-89.
8. Eelen, G., et al., Endothelial Cell Metabolism. Physiol Rev, 2018. 98(1): p. 3-58.
9. Cantelmo, A. R., et al., Inhibition of the Glycolytic Activator PFKFB3 in Endothelium Induces Tumor Vessel Normalization, Impairs Metastasis, and Improves Chemotherapy. Cancer Cell, 2016. 30(6): p. 968-985.
10. Bousseau, S., et al., Glycosylation as new pharmacological strategies for diseases associated with excessive angiogenesis. Pharmacol Ther, 2018. 191: p. 92-122.
11. Croci, D. O., et al., Glycosylation-dependent lectin-receptor interactions preserve angiogenesis in anti-VEGF refractory tumors. Cell, 2014. 156(4): p. 744-58.
12. LeCouter, J., et al., Identification of an angiogenic mitogen selective for endocrine gland endothelium. Nature, 2001. 412: p. 877-884.
13. Yu, L., et al., Interaction between bevacizumab and murine VEGF-A: a reassessment. Invest Ophthalmol Vis Sci, 2008. 49(2): p. 522-7.
14. Xin, H., et al., Evidence for Pro-angiogenic Functions of VEGF-Ax. Cell, 2016. 167(1): p. 275-284 e6.
15. Luderitz, O., et al., Identification of D-Mannosamine and Quinovosamine in Salmonella and Related Bacteria. Journal of Bacteriology, 1968. 95(2): p. 490-&.
16. Hamid, R., et al., Comparison of alamar blue and MTT assays for high through put screening. Toxicol In Vitro, 2004. 18(5): p. 703-10.
17. Raisys, V. A. and R. J. Winzler, Metabolism of exogenous D-mannosamine. J Biol Chem, 1970. 245(12): p. 3203-8.
18. Bayer, N. B., et al., Artificial and natural sialic acid precursors influence the angiogenic capacity of human umbilical vein endothelial cells. Molecules, 2013. 18(3): p. 2571-86.
19. Gonzalez, P. S., et al., Mannose impairs tumour growth and enhances chemotherapy. Nature, 2018. 563(7733): p. 719-+.
20. Uebelhoer, M. and M. L. Iruela-Arispe, Cross-talk between signaling and metabolism in the vasculature. Vascular Pharmacology, 2016. 83: p. 4-9.
21. Liao, J. K., Linking endothelial dysfunction with endothelial cell activation. Journal of Clinical Investigation, 2013. 123(2): p. 540-541.
22. Colley, K. J., A. Varki, and T. Kinoshita, Cellular Organization of Glycosylation, in Essentials of Glycobiology, rd, et al., Editors. 2015: Cold Spring Harbor (NY). p. 41-49.
23. Davis, D., et al., Isolation and Characterization of Swainsonine from Texas Locoweed (Astragalus emoryanus). Plant Physiol, 1984. 76(4): p. 972-5.
24. Pan, Y. T. and A. D. Elbein, The effect of mannosamine on the formation of lipid-linked oligosaccharides and glycoproteins in canine kidney cells. Arch Biochem Biophys, 1985. 242(2): p. 447-56.
25. Fukuda, M., Characterization of O-linked saccharides from cell surface glycoproteins. Methods Enzymol, 1989. 179: p. 17-29.
26. Xu, C. and D. T. Ng, Glycosylation-directed quality control of protein folding. Nat Rev Mol Cell Biol, 2015. 16(12): p. 742-52.
27. Cherepanova, N., S. Shrimal, and R. Gilmore, N-linked glycosylation and homeostasis of the endoplasmic reticulum. Curr Opin Cell Biol, 2016. 41: p. 57-65.
28. Preissler, S. and D. Ron, Early Events in the Endoplasmic Reticulum Unfolded Protein Response. Cold Spring Harb Perspect Biol, 2019. 11(4).
29. Griesemer, M., et al., BiP clustering facilitates protein folding in the endoplasmic reticulum. PLoS Comput Biol, 2014. 10(7): p. e1003675.
30. Frakes, A. E. and A. Dillin, The UPR(ER): Sensor and Coordinator of Organismal Homeostasis. Mol Cell, 2017. 66(6): p. 761-771.
31. Kolb, P. S., et al., The therapeutic effects of 4-phenylbutyric acid in maintaining proteostasis. Int J Biochem Cell Biol, 2015. 61: p. 45-52.
32. Ozcan, U., et al., Chemical chaperones reduce ER stress and restore glucose homeostasis in a mouse model of type 2 diabetes. Science, 2006. 313(5790): p. 1137-40.
33. Nakagawa, K., Studies targeting alpha-glucosidase inhibition, antiangiogenic effects, and lipid modification regulation: background, evaluation, and challenges in the development of food ingredients for therapeutic purposes. Biosci Biotechnol Biochem, 2013. 77(5): p. 900-8.
34. Pili, R., et al., The alpha-glucosidase I inhibitor castanospermine alters endothelial cell glycosylation, prevents angiogenesis, and inhibits tumor growth. Cancer Res, 1995. 55(13): p. 2920-6.
35. Elbein, A. D., et al., Kifunensine, a potent inhibitor of the glycoprotein processing mannosidase I. J Biol Chem, 1990. 265(26): p. 15599-605.
36. Wojtowicz, K., et al., Effect of brefeldin A and castanospermine on resistant cell lines as supplements in anticancer therapy. Oncol Rep, 2016. 35(5): p. 2896-906.
37. Whitby, K., et al., Castanospermine, a potent inhibitor of dengue virus infection in vitro and in vivo. J Virol, 2005. 79(14): p. 8698-706.
38. Qin, S., et al., TBC1D3 regulates the payload and biological activity of extracellular vesicles that mediate tissue repair. FASEB J, 2019. 33(5): p. 6129-6139.
39. Tseng, C. W., et al., Increased Susceptibility of Humanized NSG Mice to Panton-Valentine Leukocidin and Staphylococcus aureus Skin Infection. PLoS Pathog, 2015. 11(11): p. e1005292.
40. Hellingman, A. A., et al., Variations in surgical procedures for hind limb ischaemia mouse models result in differences in collateral formation. Eur J Vasc Endovasc Surg, 2010. 40(6): p. 796-803.
41. Nowak-Sliwinska, P., et al., Consensus guidelines for the use and interpretation of angiogenesis assays. Angiogenesis, 2018. 21(3): p. 425-532.
42. Schlickeiser, S., et al., Control of TNF-induced dendritic cell maturation by hybrid-type N-glycans. J Immunol, 2011. 186(9): p. 5201-11.
43. Ungerleider, J. L., et al., Extracellular Matrix Hydrogel Promotes Tissue Remodeling, Arterio genesis, and Perfusion in a Rat Hindlimb Ischemia Model. JACC Basic Transl Sci, 2016. 1(1-2): p. 32-44.
44. Kim, J. A., et al., Muscle-derived Gr 1 (dim)CD11b(+) cells enhance neovascularization in an ischemic hind limb mouse model. Blood, 2010. 116(9): p. 1623-6.
45. Yoneyama, T., et al., Distribution of mannosamine and mannosaminuronic acid among cell walls of Bacillus species. Journal of Bacteriology, 1982. 149(1): p. 15-21.
46. Monaco, F. and J. Robbins, Incorporation of N-acetylmannosamine and N-acetylglucosamine into thyroglobulin in rat thyroid in vitro. J Biol Chem, 1973. 248(6): p. 2072-7.
47. Onoda, T., et al., Antitumor activity of D-mannosamine in vitro: different sensitivities among human leukemia cell lines possessing T-cell properties. Cancer Res, 1982. 42(7): p. 2867-71.
48. Chen, Y. J., et al., Hexosamine-Induced TGF-beta Signaling and Osteogenic Differentiation of Dental Pulp Stem Cells Are Dependent on N-Acetylglucosaminyltransferase V. Biomed Res Int, 2015. 2015: p. 924397.
49. Patwari, P., et al., Mannosamine inhibits aggrecanase-mediated changes in the physical properties and biochemical composition of articular cartilage. Arch Biochem Biophys, 2000. 374(1): p. 79-85.
50. Salton, M. R., Chemistry and Function of Amino Sugars and Derivatives. Annu Rev Biochem, 1965. 34: p. 143-74.
51. Farley, J. R. and P. Magnusson, Effects of tunicamycin, mannosamine, and other inhibitors of glycoprotein processing on skeletal alkaline phosphatase in human osteoblast-like cells. Calcif Tissue Int, 2005. 76(1): p. 63-74.
52. Alonso-Sande, M., et al., Development of PLGA-mannosamine nanoparticles as oral protein carriers. Biomacromolecules, 2013. 14(11): p. 4046-52.
53. Liu, J., et al., Synthesis and high-throughput screening of N-acetyl-beta-hexosaminidase inhibitor libraries targeting osteoarthritis. J Org Chem, 2004. 69(19): p. 6273-83.
54. Estrada-Mondaca, S., L. A. Delgado-Bustos, and O. T. Ramirez, Mannosamine supplementation extends the N-acetylglucosaminylation of recombinant human secreted alkaline phosphatase produced in Trichoplusia ni (cabbage looper) insect cell cultures. Biotechnol Appl Biochem, 2005. 42(Pt 1): p. 25-34.
55. Sevlever, D. and T. L. Rosenberry, Mannosamine inhibits the synthesis of putative glycoinositol phospholipid anchor precursors in mammalian cells without incorporating into an accumulated intermediate. J Biol Chem, 1993. 268(15): p. 10938-45.
56. Ralton, J. E., et al., The mechanism of inhibition of glycosylphosphatidylinositol anchor biosynthesis in Trypanosoma brucei by mannosamine. J Biol Chem, 1993. 268(32): p. 24183-9.
57. Li, C., et al., Endothelial AMPK activation induces mitochondrial biogenesis and stress adaptation via eNOS-dependent mTORC1 signaling. Nitric Oxide, 2016. 55-56: p. 45-53.
58. Shang, J., et al., Extension of lipid-linked oligosaccharides is a high-priority aspect of the unfolded protein response: endoplasmic reticulum stress in Type I congenital disorder of glycosylation fibroblasts. Glycobiology, 2002. 12(5): p. 307-17.
59. Lau, K. S., et al., Complex N-glycan number and degree of branching cooperate to regulate cell proliferation and differentiation. Cell, 2007. 129(1): p. 123-34.
60. Chandler, K. B., C. E. Costello, and N. Rahimi, Glycosylation in the Tumor Microenvironment: Implications for Tumor Angiogenesis and Metastasis. Cells, 2019. 8(6).
61. Croci, D. O. and G. A. Rabinovich, Linking tumor hypoxia with VEGFR2 signaling and compensatory angiogenesis: Glycans make the difference. Oncoimmunology, 2014. 3: p. e29380.
62. Martinez, M. R., et al., Stress-induced O-GlcNAcylation: an adaptive process of injured cells. Biochem Soc Trans, 2017. 45(1): p. 237-249.
63. Ferrara, N. and R. S. Kerbel, Angiogenesis as a therapeutic target. Nature, 2005. 438: p. 967-974.
64. Gan, L. M., et al., Intradermal delivery of modified mRNA encoding VEGF-A in patients with type 2 diabetes. Nat Commun, 2019. 10(1): p. 871.
65. Ackermann, M., et al., Pulmonary Vascular Endothelialitis, Thrombosis, and Angiogenesis in Covid-19. N Engl J Med, 2020. 383(2): p. 120-128.
66. Moreira-Neto, C. A., et al., Choriocapillaris Loss in Advanced Age Related Macular Degeneration. J Ophthalmol, 2018. 2018: p. 8125267.
67. Ferrara, N., et al., Transport epithelial characteristics of cultured bovine pituitary follicular cells. Am J Physiol, 1987. 252(3 Pt 1): p. E304-12.
68. Liang, W., Q. Li, and N. Ferrara, Metastatic growth instructed by neutrophil-derived transferrin. Proc Natl Acad Sci USA, 2018. 115(43): p. 11060-11065.
69. Hardy, M. R., R. R. Townsend, and Y. C. Lee, Monosaccharide analysis of glycoconjugates by anion exchange chromatography with pulsed amperometric detection. Anal Biochem, 1988. 170(1): p. 54-62.
70. Marcellin, E., et al., Quantitative analysis of intracellular sugar phosphates and sugar nucleotides in encapsulated streptococci using HPAEC-PAD. Biotechnol J, 2009. 4(1): p. 58-63.

What is claimed is:

1. A method of treating an ischemic condition in a subject, the method comprising administering to the subject in need thereof an effective amount of hexosamine D-mannosamine (ManN).

2. The method of claim 1, wherein the administration is effective to promote endothelial cell proliferation and angiogenesis in the subject.

3. The method of claim 1, wherein the method further comprises administering to the subject in need thereof an effective amount of an N-glycosylation inhibitor.

4. The method of claim 1, wherein the method further comprises administering to the subject in need thereof an effective amount of VEGF.

5. The method of claim 1, wherein the ischemic condition is caused by a disease or a trauma.

6. The method of claim 1, wherein the administration is intravenous, intraperitoneal, or intravitreal.

7. A method of inducing angiogensis in a subject, the method comprising administering to the subject in need thereof an effective amount of hexosamine D-mannosamine (ManN).

8. The method of claim 7, wherein the administration is effective to reduce ischemia in the subject.

9. The method of claim 7, wherein the method further comprises administering to the subject in need thereof an effective amount of an N-glycosylation inhibitor.

10. The method of claim 7, wherein the method further comprises administering to the subject in need thereof an effective amount of VEGF.

11. The method of claim 7, wherein the subject is in need of inducing angiogenesis is due to an ischemic condition caused by a disease or a trauma.

12. The method of claim 7, wherein the administration is intravenous, intraperitoneal, or intravitreal.

13. A method of inhibiting protein glycosylation in a cell, the method comprising administering to the cell an effective amount of hexosamine D-mannosamine (ManN).

14. The method of claim 13, wherein the administration is in vivo.

15. The method of claim 13, wherein the administration is ex vivo.

16. The method of claim 13, wherein the administration is effective to stimulate EC proliferation and angiogenesis.

17. The method of claim 13, wherein the administration is effective to activate JNK and an unfolded protein response caused by ER stress.

18. The method of claim 13, wherein the administration is effective to induce changes in N-glycan and O-glycan profiles.
